(19) United States
(12) Patent Application Publication
(10) Pub. No.: US 2008/0292581 A1
(43) Pub. Date: Nov. 27, 2008

(54) Interleukin-11 Fusion Proteins

(75) Inventors: Monika Kroz, Altheim-Waldhausen (DE); Gerhard Dickneite, Marburg (DE); Hans-Peter Hauser, Marburg (DE); Thomas Weimer, Gladenbach (DE); Darrell Sleep, Nottingham (GB)

Correspondence Address: FOX ROTHSCHILD LLP, 1101 MARKET STREET, SUITE 2600, PHILADELPHIA, PA, 19107-2950, US

(73) Assignee: Delta Biotechnology Limited, Nottingham (GB)

(21) Appl. No.: 10/584,438

(22) PCT Filed: Dec. 3, 2004
(86) PCT No.: PCT/GB2004/005091

§ 371 Date: Jan. 29, 2008

(30) Foreign Application Priority Data
Dec. 3, 2003 (EP) 03027770.1

(51) Int. Cl. C07K 14/54; C12N 15/24; C12N 15/81; C12N 15/85; C12P 21/02; C12N 5/10; C12N 1/19; A61P 1/00; A61P 3/00; A61K 38/20

(52) U.S. Cl. 424/85.2; 530/351; 536/23.4; 435/254.2; 435/69.52; 435/325

(57) ABSTRACT

The invention relates to proteins comprising Interleukin 11 (IL-11) (including, but not limited to, fragments and variants thereof), which exhibit thrombopoietic or antiinflammatory properties, fused to albumin (including, but not limited to fragments or variants of albumin). These fusion proteins are herein collectively referred to as “albumin fusion proteins of the invention”. These fusion proteins exhibit extended shelf-life and/or pharmacokinetic properties and/or extended or therapeutic activity. The invention encompasses therapeutic albumin fusion proteins, compositions, pharmaceutical compositions, formulations and kits. The invention also encompasses nucleic acid molecules encoding the albumin fusion proteins of the invention, as well as vectors containing these nucleic acids, host cells transformed with these nucleic acids and vectors, and methods of making the albumin fusion proteins of the invention using these nucleic acids, vectors, and/or host cells. The invention also relates to compositions and methods for treatment or prophylaxis of thrombocytopenia or inflammatory diseases.

### FIELD OF THE INVENTION

The invention relates to novel compositions for treatment or prophylaxis of thrombocytopenia, von Willebrand disease (vWD) or inflammatory diseases, such as inflammatory bowel disease (IBD).

### BACKGROUND OF THE INVENTION

### Physiological Function of IL-11

Interleukin eleven (IL-11) is a hematopoietic cytokine that promotes megakaryocytopoiesis and thrombocytopoiesis by stimulating the proliferation of primitive stem cells, multipotent and committed progenitor cells (synergistic with other hematopoietic growth factors like IL-3, IL-6, GM-CSF) resulting in megakaryocyte maturation and increased platelet production. Due to the activity described above it stimulates erythropoiesis as a side effect but shows little effect on neutrophil proliferation. It also has trophic effects on intestinal mucosa cells. Furthermore IL-11 induces the secretion of acute phase proteins (ferritin, haptoglobin, CRP, fibrinogen) by hepatocytes. It also shows antiinflammatory properties by inhibiting macrophage and T cell effector function. It inhibits the production of TNFα, IL-1β, IL-12, IL-6 and NO from activated macrophages in-vitro. Physiologically, IL-11 is produced by a variety of stromal cells, like fibroblasts, epithelial cells, chondrocytes and osteoblasts. In normal individuals it is generally undetectable in plasma. Degradation and elimination of IL-11 is yet poorly understood.

### Biochemical Characteristics of IL-11

IL-11 is a 19 kDa polypeptide consisting of 178 amino acids (AA), plus 21 AA secretory leader sequence, which does not contain potential glycosylation residues, disulphide bonds or other posttranslational modifications, and has a close similarity to IL-6. It binds to a multimeric receptor complex which contains an IL-11 specific α-receptor subunit and a promiscuous β subunit (gp130).

Recombinant human IL-11 (Oprelvekin, Neumega® by Genetics Institute/Wyeth) is a 2-178-interleukin-11 produced in E. coli lacking the amino terminal proline. This can be necessary to achieve secretion from the host cell but reportedly does not affect the activity of the cytokine.

### Chemotherapy-Induced Thrombocytopenia

Thrombocytopenia is a significant problem for patients receiving prolonged or aggressive chemotherapy for malignancies. For some chemotherapeutic agents, such as Carboplatin, it represents the predominant, dose-limiting toxicity and acts cumulatively.

Currently, platelet transfusion is the standard treatment for thrombocytopenia However, platelet transfusions are expensive and associated with a significant risk of alloimmunisation and transmission of blood-borne diseases. Approximately 5 to 30% of platelet transfusions are associated with usually febrile, non-hemolytic reactions. Also, platelets are a limited resource with a shelf life of 5 days only. Thus, agents that promote platelet production are an attractive alternative to platelet transfusions for the prevention or treatment of thrombocytopenia Recombinant human IL-11 (Oprelvekin, Neumega®) was approved in 1997 in the US for the secondary prophylaxis of chemotherapy-induced thrombocytopenia. Further potential indications include inflammatory bowel disease (IBD), Crohn's disease, colitis ulcerosa, psoriasis and von Willebrand's disease.

### Expected Advantages of Fusing IL11 to Albumin

Safe and effective treatment of thrombocytopenia remains an unmet medical need. Recombinant human IL-11 (Oprelvekin, Neumega®) has a very short half-life of 6.9 hrs in humans and at the same time a narrow therapeutic window.

Prolongation of plasma-half-life and increased bioavailability through albumin-fusion are expected to result in an improved safety and efficacy profile. This means that therapeutic plasma levels can be achieved and maintained with lower doses and/or longer dose intervals, avoiding peak levels above the toxic threshold. Currently, Neumega® is the only drug licensed for the treatment of chemotherapy-induced thrombocytopenia, representing a field with a high unmet medical need. Neumega® shows a high incidence of toxic effects, such as edema and cardiovascular irregularities, combined with low efficacy especially in severe cases of thrombocytopenia.

### SUMMARY OF THE INVENTION

The invention relates to proteins comprising IL-11 fused to albumin or fragments or variants thereof. These fusion proteins are herein collectively referred to as “albumin fusion proteins of the invention.” These fusion proteins of the invention exhibit extended in vivo half-life and/or extended therapeutic activity as compared to unfused IL11.

The invention encompasses therapeutic albumin fusion proteins, compositions, pharmaceutical compositions, formulations and kits. The invention also encompasses nucleic acid molecules encoding the albumin fusion proteins of the invention, as well as vectors containing these nucleic acids, host cells transformed with these nucleic acids and vectors, and methods of making the albumin fusion proteins of the invention using these nucleic acids, vectors, and/or host cells.

The invention also relates to compositions and methods for therapy and prevention of thrombocytopenia. The invention further relates to compositions and methods for antiinflammatory therapy and prevention. Also, the invention relates to compositions and methods for therapy and prevention of von Willebrand's disease.

### BRIEF DESCRIPTION OF THE DRAWINGS

FIG. 1. IL-11 plasma concentration after intravenous administration of rhIL-11 or C-terminal IL-11-albumin fusion to rabbits

FIG. 2. IL-11 plasma concentration after subcutaneous administration of rhIL-11 or C-terminal IL-11-albumin fusion to rabbits

FIG. 3. IL-11 plasma concentration after intravenous administration of rhIL-11 or N-terminal IL-11-albumin fusion to rats

FIG. 4. IL-11 plasma concentration after subcutaneous administration of rhIL-11 or N-terminal IL-11-albumin fusion to rats

FIG. 5. Course of platelet levels after treatment of naive rats with IL-11

FIG. 6. Course of platelet levels after treatment of rats under chemotherapy with IL-11

FIG. 7. Development of body weight (in % of baseline) after IL-11 application in a mouse model for IBD

FIG. 8. Visual observation score (diarrhoea and gross rectal bleeding) after IL-11 application in a mouse model for IBD

FIG. 9. Colon length after IL-11 application in a mouse model for IBD

FIG. 10. Histological disease score after IL-11 application in a mouse model for IBD

FIG. 11 SDS gel and Western blots of various compounds of the invention.

### DETAILED DESCRIPTION OF THE INVENTION

The present invention relates to fusion proteins comprising albumin coupled to IL-11. Such peptides include, but are not limited to, peptides binding to the gp130 receptor complex. These peptides include IL-11, or fragments or variants thereof, which have thrombopoietic or antiinflammatory properties.

The terms “protein” and “peptide” as used herein are non-limiting and include proteins and polypeptides as well as peptides.

Furthermore, chemical entities may be covalently attached to the fusion proteins of the invention or used in combinations to enhance a biological activity or to modulate a biological activity.

The albumin fusion proteins of the present invention are expected to prolong the half-life of IL-11 in vivo. The in vitro or in vivo half-life of said albumin-fused peptide/protein is extended 2-fold, or 5-fold, or more, over the half-life of the peptide/protein lacking the linked albumin. Furthermore, the albumin fusion proteins of the present invention are expected to reduce the frequency of the dosing schedule of the therapeutic peptide. The dosing schedule frequency is reduced by at least one-quarter, or by at least one-half, or more, as compared to the frequency of the dosing schedule of the therapeutic peptide lacking the linked albumin.

The albumin fusion proteins of the present invention are expected to prolong the shelf-life of the peptide, and/or stabilize the peptide and/or its activity in solution (or in a pharmaceutical composition) in vitro and/or in vivo. These albumin-fusion proteins, which may be therapeutic agents, are expected to reduce the need to formulate protein solutions with large excesses of carrier proteins (such as albumin, unfused) to prevent loss of proteins due to factors such as non-specific binding. An increased half-life is defined as a half-life that is at least 2 times higher (preferably at least 5, 10, 20 or 30 times higher) than that of the unfused IL-11 compound, when measured over the first 24 hours after sub-cutaneous injection according to Example 4 below, in male Wistar rats aged 6 months.

The present invention also encompasses nucleic acid molecules encoding the albumin fusion proteins as well as vectors containing these nucleic acids, host cells transformed with these nucleic acids vectors, and methods of making the albumin fusion proteins of the invention using these nucleic acids, vectors, and/or host cells. The present invention further includes transgenic organisms modified to contain the nucleic acid molecules of the invention, optionally modified to express the albumin fusion proteins encoded by the nucleic acid molecules.

The present invention also encompasses pharmaceutical formulations comprising an albumin fusion protein of the invention and a pharmaceutically acceptable diluent or carrier. Such formulations may be in a kit or container. Such kit or container may be packaged with instructions pertaining to the extended shelf-life of the protein. Such formulations may be used in methods of preventing, treating, ameliorating thrombocytopenia or inflammatory diseases, such as inflammatory bowel disease, psoriasis, rheumatoid arthritis, von Willebrand's disease, etc., or a related disorder in a patient, such as a mammal, or a human, comprising the step of administering the pharmaceutical formulation to the patient.

The invention also encompasses a method for potentially minimizing side effects (e.g., injection site reaction, increase of plasma volume, arrhythmia, headache, nausea, fever, rash, asthenia, diarrhoea, dizziness, allergic reactions) associated with the treatment of a mammal with cytokines in moderately higher concentrations comprising administering an albumin-fused cytokine of the invention to said mammal.

The present invention encompasses a method of preventing, treating or ameliorating thrombocytopenia or inflammatory diseases, such as inflammatory bowel disease, psoriasis, rheumatoid arthritis, etc., comprising administering to a mammal, in which such prevention treatment or amelioration is desired, an albumin fusion protein of the invention that comprises a IL-11 peptide/protein (or fragment or variant thereof) in an amount effective to treat, prevent or ameliorate the disease or disorder. In the present invention, the IL-11 is also called the “therapeutic protein”.

The present invention encompasses albumin fusion proteins comprising IL-11 (including fragments and variants thereof) fused to albumin or multiple copies of albumin (including fragments and variants thereof).

The present invention also encompasses a method for extending the half-life of IL-11 in a mammal. The method entails linking IL-11 to an albumin to form albumin-fused IL-11 and administering the albumin-fused IL-11 to a mammal. Typically, the half-life of the albumin-fused IL-11 may be extended by at least 2-fold, 5-fold, 10-fold, 20-fold, 30-fold, 40-fold or at least 50-fold over the half-life of IL-11 lacking the linked albumin.

Exemplified herein are fusion proteins comprising albumin fused to IL-11. The present invention also includes an improved method of manufacturing a therapeutic moiety as compared to what is available in the art. For example, the present invention provides an enhanced means of manufacturing a protein with the active moiety IL-11 Various aspects of the present invention are discussed in further detail below

### Albumin

The terms human serum albumin (HSA) and human albumin (HA) are used interchangeably herein. The terms, “albumin and “serum albumin” are broader, and encompass human serum albumin (and fragments and variants thereof) as well as albumin from other species (and fragments and variants thereof).

As used herein, “albumin” refers collectively to albumin protein or amino acid sequence, or an albumin fragment or variant, having one or more functional activities (e.g., biological activities) of albumin. In particular, “albumin” refers to human albumin or fragments thereof (see EP 201 239, EP 322 094 and WO 97/24445) especially the mature form of human albumin as shown in Table 1 and SEQ ID NO:18 of WO 03/066824 and WO 01/79480, or residues 202 to 762 of SEQ ID No: 17 herein, or albumin from other vertebrates or fragments thereof, or analogs or variants of these molecules or fragments thereof (for example the modified albumins of WO95/23857).

The human serum albumin protein used in the albumin fusion proteins of the invention may contain one or both of the following sets of point mutations with reference to SEQ ID NO:18: Leu-407 to Ala, Leu-408 to Val, Val-409 to Ala, and Arg-410 to Ala; or Arg-410 to Ala, Lys-413 to Gln, and Lys-414 to Gln (see, e.g., International Publication No. WO95/23857, hereby incorporated in its entirety by reference herein). In other embodiments, albumin fusion proteins of the invention that contain one or both of above-described sets of point mutations have improved stability/resistance to yeast Yap3p proteolytic cleavage, allowing increased production of recombinant albumin fusion proteins expressed in yeast host cells.

As used herein, a portion of albumin sufficient to prolong or extend the in vivo half-life, therapeutic activity, or shelf-life of the Therapeutic protein refers to a portion of albumin sufficient in length or structure to stabilize, prolong or extend the in vivo half-life, therapeutic activity or shelf-life of the Therapeutic protein portion of the albumin fusion protein compared to the in vivo half-life, therapeutic activity, or shelf-life of the Therapeutic protein in the non-fusion state. The albumin portion of the albumin fusion proteins may comprise the full length of the HA sequence as described above, or may include one or more fragments thereof that are capable of stabilizing or prolonging the therapeutic activity. Such fragments may be of 10 or more amino acids in length or may include about 15, 20, 25, 30, 50, 100, 150 or more contiguous amino acids from the HA sequence or may include part or all of specific domains of HA.

The albumin portion of the albumin fusion proteins of the invention may be a variant of normal HA. The Therapeutic protein portion of the albumin fusion proteins of the invention may also be variants of the Therapeutic proteins as described herein. The term “variants” includes insertions, deletions and substitutions, either conservative or non conservative, where such changes do not substantially alter one or more of the oncotic, useful ligand-binding and non-immunogenic properties of albumin, or the active site, or active domain which confers the therapeutic activities of the Therapeutic proteins.

In particular, the albumin fusion proteins of the invention may include naturally occurring polymorphic variants of human albumin and fragments of human albumin, for example those fragments disclosed in EP 322 094 (namely HA (1-n), where n is 369 to 419). The albumin may be derived from any vertebrate, especially any mammal, for example human, cow, sheep or pig. Non-mammalian albumins include, but are not limited to, hen and salmon. The albumin portion of the albumin fusion protein may be from a different animal than the Therapeutic protein portion.

Generally speaking, an HA fragment or variant will be at least 100 amino acids long, optionally at least 150 amino acids long. The HA variant may consist of or alternatively comprise at least one whole domain of HA, for example domains 1 (amino acids 1-194 of SEQ ID NO:18), 2 (amino acids 195-387 of SEQ ID NO:18), 3 (amino acids 388-585 of SEQ ID NO:18), 1+2 (1-387 of SEQ ID NO:18), 2+3 (195-585 of SEQ ID NO:18) or 1+3 (amino acids 1-194 of SEQ ID NO:18+ amino acids 388-585 of SEQ ID NO:18). Each domain is itself made up of two homologous subdomains namely 1-105, 120-194, 195-291, 316-387, 388-491 and 512-585, with flexible inter-subdomain linker regions comprising residues Lys106 to Glu119, Glu292 to Val315 and Glu492 to Ala511.

The albumin portion of an albumin fusion protein of the invention may comprise at least one subdomain or domain of HA or conservative modifications thereof. If the fusion is based on subdomains, some or all of the adjacent linkers may optionally be used to link to the Therapeutic protein moiety.

### Albumin Fusion Proteins

The present invention relates generally to albumin fusion proteins and methods of treating, preventing or ameliorating diseases or disorders. As used herein, “albumin fusion protein” refers to a protein formed by the fusion of at least one molecule of albumin (or a fragment or variant thereof) to at least one molecule of a Therapeutic protein (or fragment or variant thereof). An albumin fusion protein of the invention comprises at least a fragment or variant of a Therapeutic protein and at least a fragment or variant of human serum albumin, which are associated with one another, such as by genetic fusion (i.e., the albumin fusion protein is generated by translation of a nucleic acid in which a polynucleotide encoding all or a portion of a Therapeutic protein is joined in-frame with a polynucleotide encoding all or a portion of albumin) to one another. The Therapeutic protein and albumin protein, once part of the albumin fusion protein, may be referred to as a “portion”, “region” or “moiety” of the albumin fusion protein.

In one embodiment, the invention provides an albumin fusion protein comprising, or alternatively consisting of, a Therapeutic protein and a serum albumin protein. In other embodiments, the invention provides an albumin fusion protein comprising, or alternatively consisting of, a biologically active and/or therapeutically active fragment of a Therapeutic protein and a serum albumin protein. In other embodiments, the invention provides an albumin fusion protein comprising, or alternatively consisting of, a biologically active and/or therapeutically active variant of a Therapeutic protein and a serum albumin protein. In further embodiments, the serum albumin protein component of the albumin fusion protein is the mature portion of serum albumin.

In further embodiments, the invention provides an albumin fusion protein comprising, or alternatively consisting of, a Therapeutic protein, and a biologically active and/or therapeutically active fragment of serum albumin. In further embodiments, the invention provides an albumin fusion protein comprising, or alternatively consisting of, a Therapeutic protein and a biologically active and/or therapeutically active variant of serum albumin. In some embodiments, the Therapeutic protein portion of the albumin fusion protein is the mature portion of the Therapeutic protein.

In further embodiments, the invention provides an albumin fusion protein comprising, or alternatively consisting of, a biologically active and/or therapeutically active fragment or variant of a Therapeutic protein and a biologically active and/or therapeutically active fragment or variant of serum albumin. In some embodiments, the invention provides an albumin fusion protein comprising, or alternatively consisting of, the mature portion of a Therapeutic protein and the mature portion of serum albumin.

In one embodiment, the albumin fusion protein comprises HA as the N-terminal portion, and a Therapeutic protein as the C-terminal portion. Alternatively, an albumin fusion protein comprising HA as the C-terminal portion, and a Therapeutic protein as the N-terminal portion may also be used.

In other embodiments, the albumin fusion protein has a Therapeutic protein fused to both the N-terminus and the C-terminus of albumin. In one embodiment, the Therapeutic proteins fused at the N- and C-termini are the same Therapeutic proteins. In another embodiment, the Therapeutic proteins fused at the N- and C-termini are different Therapeutic proteins. In another embodiment, the Therapeutic proteins fused at the N- and C-termini are different Therapeutic proteins which may be used to treat or prevent the same disease, disorder, or condition. In another embodiment, the Therapeutic proteins fused at the N- and C-termini are different Therapeutic proteins which may be used to treat or prevent diseases or disorders which are known in the art to commonly occur in patients simultaneously.

In addition to albumin fusion protein in which the albumin portion is fused N-terminal and/or C-terminal of the Therapeutic protein portion, albumin fusion proteins of the invention may also be produced by inserting the Therapeutic protein or peptide of interest into an internal region of HA. For instance, within the protein sequence of the HA molecule a number of loops or turns exist between the end and beginning of α-helices, which are stabilized by disulphide bonds. The loops, as determined from the crystal structure of HA (PDB identifiers 1AO6, 1BJ5, 1BKE, 1BM0, 1E7E to 1E7I and 1UOR) for the most part extend away from the body of the molecule. These loops are useful for the insertion, or internal fusion, of therapeutically active peptides, particularly those requiring a secondary structure to be functional, or Therapeutic proteins, to essentially generate an albumin molecule with specific biological activity.

Loops in human albumin structure into which peptides or polypeptides may be inserted to generate albumin fusion proteins of the invention include: Val54-Asn61, Thr76-Asp89, Ala92-Glu100, Gln170-Ala176, His247-Glu252, Glu266-Glu277, Glu280-His288, Ala362-Glu368, Lys439-Pro447, Val462-Lys475, Thr478-Pro486, and Lys560-Thr566. In other embodiments, peptides or polypeptides are inserted into the Val54-Asn61, Gln170-Ala176, and/or Lys560-Thr566 loops of mature human albumin (SEQ ID NO:18).

Peptides to be inserted may be derived from either phage display or synthetic peptide libraries screened for specific biological activity or from the active portions of a molecule with the desired function. Additionally, random peptide libraries may be generated within particular loops or by insertions of randomized peptides into particular loops of the HA molecule and in which all possible combinations of amino acids are represented.

Such library(s) could be generated on HA or domain fragments of HA by one of the following methods:

(a) randomized mutation of amino acids within one or more peptide loops of HA or HA domain fragments. Either one, more or all the residues within a loop could be mutated in this manner;

(b) replacement of, or insertion into one or more loops of HA or HA domain fragments (i.e., internal fusion) of a randomized peptide(s) of length Xₙ (where X is an amino acid and n is the number of residues;

(c) N-, C- or N- and C-terminal peptide/protein fusions in addition to (a) and/or (b).

The HA or HA domain fragment may also be made multifunctional by grafting the peptides derived from different screens of different loops against different targets into the same HA or HA domain fragment.

Examples of peptides inserted into a loop of human serum albumin are Therapeutic protein peptides or peptide fragments or peptide variants thereof. For example, peptides inserted into a loop of human serum albumin may include T-20 and/or T-1249 peptide or peptide fragments or peptide variants thereof. More particularly, the invention encompasses albumin fusion proteins which comprise peptide fragments or peptide variants of at least 7 at least 8, at least 9, at least 10, at least 11, at least 12, at least 13, at least 14, at least 15, at least 20, at least 25, at least 30, at least 35, or at least 40 amino acids in length inserted into a loop of human serum albumin. The invention also encompasses albumin fusion proteins which comprise peptide fragments or peptide variants having at least 7 at least 8, at least 9, at least 10, at least 11, at least 12, at least 13, at least 14, at least 15, at least 20, at least 25, at least 30, at least 35, or at least 40 amino acids fused to the N-terminus of human serum albumin. The invention also encompasses albumin fusion proteins which comprise peptide fragments or peptide variants having at least 7, at least 8, at least 9, at least 10, at least 11, at least 12, at least 13, at least 14, at least 15, at least 20, at least 25, at least 30, at least 35, or at least 40 amino acids fused to the C-terminus of human serum albumin.

Generally, the albumin fusion proteins of the invention may have one HA-derived region and one Therapeutic protein-derived region. Multiple regions of each protein, however, may be used to make an albumin fusion protein of the invention. Similarly, more than one Therapeutic protein may be used to make an albumin fusion protein of the invention. For instance, a Therapeutic protein may be fused to both the N- and C-terminal ends of the HA. In such a configuration, the Therapeutic protein portions may be the same or may be different Therapeutic protein molecules. The structure of bifunctional albumin fusion proteins may be represented as: X-HA-Y or Y-HA-X or X-Y-HA or HA-X-Y or HA-Y-X-HA or HA-X-X-HA or HA Y-Y-HA or HA-X-HA-Y or X-HA-Y-HA or multiple combinations and/or inserting X and/or Y within the HA sequence at any location

Bi- or multi-functional albumin fusion proteins may be prepared in various ratios depending on function, half-life etc. Bi- or multi-functional albumin fusion proteins may also be prepared to target the Therapeutic protein portion of a fusion to a target organ or cell type via protein or peptide at the opposite terminus of HA.

As an alternative to the fusion of known therapeutic molecules, the peptides may be obtained by screening libraries constructed as fusions to the N-, C- or N- and C-termini of HA, or domain fragment of HA, of typically 6, 8, 12, 20 or 25 or Xₙ (where X is an amino acid (aa) and n equals the number of residues) randomized amino acids, and in which all possible combinations of amino acids are represented. A particular advantage of this approach is that the peptides may be selected in situ on the HA molecule and the properties of the peptide would therefore be as selected for rather than, potentially, modified as might be the case for a peptide derived by any other method then being attached to HA.

Additionally, the albumin fusion proteins of the invention may include a linker peptide between the fused portions to provide greater physical separation between the moieties and thus maximize the accessibility of the Therapeutic protein portion, for instance, for binding to its cognate receptor. The linker peptide may consist of amino acids such that it is flexible or more rigid.

Therefore, as described above, the albumin fusion proteins of the invention may have the following formula R2-R1; R1-R2; R2-R1-R2; R2-L-R1-L-R2; R1-L-R2; R2-L-R1; or R1-L-R2-L-R1, wherein R1 is at least one Therapeutic protein, peptide or polypeptide sequence (including fragments or variants thereof), and not necessarily the same Therapeutic protein, L is a linker and R2 is a serum albumin sequence (including fragments or variants thereof). Exemplary linkers include (GGGGS)_{N} (SEQ ID NO:8) or (GGGS)_{N} (SEQ ID NO:9) or (GGS)_{N}, wherein N is an integer greater than or equal to 1 and wherein G represents glycine and S represents serine. When R1 is two or more Therapeutic proteins, peptides or polypeptide sequence, these sequences may optionally be connected by a linker.

In further embodiments, albumin fusion proteins of the invention comprising a Therapeutic protein have extended shelf-life or in vivo half-life or therapeutic activity compared to the shelf-life or in vivo half-life or therapeutic activity of the same Therapeutic protein when not fused to albumin. Shelf-life typically refers to the time period over which the therapeutic activity of a Therapeutic protein in solution or in some other storage formulation, is stable without undue loss of therapeutic activity. Many of the Therapeutic proteins are highly labile in their unfused state. As described below, the typical shelf-life of these Therapeutic proteins is markedly prolonged upon incorporation into the albumin fusion protein of the invention.

Albumin fusion proteins of the invention with “prolonged” or “extended” shelf-life exhibit greater therapeutic activity relative to a standard that has been subjected to the same storage and handling conditions. The standard may be the unfused full-length Therapeutic protein. When the Therapeutic protein portion of the albumin fusion protein is an analogue, a variant, or is otherwise altered or does not include the complete sequence for that protein, the prolongation of therapeutic activity may alternatively be compared to the unfused equivalent of that analogue, variant, altered peptide or incomplete sequence. As an example, an albumin fusion protein of the invention may retain greater than about 100% of the therapeutic activity, or greater than about 105%, 110%, 120%, 130%, 150% or 200% of the therapeutic activity of a standard when subjected to the same storage and handling conditions as the standard when compared at a given time point. However, it is noted that the therapeutic activity depends on the Therapeutic protein's stability, and may be below 100%.

Shelf-life may also be assessed in terms of therapeutic activity remaining after storage, normalized to therapeutic activity when storage began. Albumin fusion proteins of the invention with prolonged or extended shelf-life as exhibited by prolonged or extended therapeutic activity may retain greater than about 50% of the therapeutic activity, about 60%, 70%, 80%, or 90% or more of the therapeutic activity of the equivalent unfused Therapeutic protein when subjected to the same conditions.

### Therapeutic Proteins

As stated above, an albumin fusion protein of the invention comprises at least a fragment or variant of a therapeutic protein and at least a fragment or variant of human serum albumin, which are associated with one another by genetic fusion.

As used herein, “Therapeutic protein” refers to IL-11, or fragments or variants thereof, having one or more therapeutic and/or biological activities. Thus an albumin fusion protein of the invention may contain at least a fragment or variant of a Therapeutic protein. Additionally, the term “therapeutic protein” may refer to the endogenous or naturally occurring correlate of a therapeutic protein. Variants include mutants, analogs, and mimetics, as well as homologues, including the endogenous or naturally occurring correlates of a therapeutic protein.

By a polypeptide displaying a “therapeutic activity” or a protein that is “therapeutically active” is meant a polypeptide that possesses one or more known biological and/or therapeutic activities associated with a therapeutic protein such as one or more of the therapeutic proteins described herein or otherwise known in the art. As a non-limiting example, a “therapeutic protein” is a protein that is useful to treat, prevent or ameliorate a disease, condition or disorder.

As used herein, “therapeutic activity” or “activity” may refer to an activity whose effect is consistent with a desirable therapeutic outcome in humans, or to desired effects in non-human mammals or in other species or organisms. Therapeutic activity may be measured in vivo or in vitro. For example, a desirable effect may be assayed in cell culture. Such in vitro or cell culture assays are commonly available for many Therapeutic proteins as described in the art.

Therapeutic proteins corresponding to a therapeutic protein portion of an albumin fusion protein of the invention may be modified by the attachment of one or more oligosaccharide groups. The modification, referred to as glycosylation, can dramatically affect the physical properties of proteins and can be important in protein stability, secretion, and localization. Such modifications are described in detail in WO 03/066824 and WO 01/79480, which are incorporated herein by reference

Therapeutic proteins corresponding to a Therapeutic protein portion of an albumin fusion protein of the invention, as well as analogs and variants thereof, may be modified so that glycosylation at one or more sites is altered as a result of manipulation(s) of their nucleic acid sequence, by the host cell in which they are expressed, or due to other conditions of their expression. For example, glycosylation isomers may be produced by abolishing or introducing glycosylation sites, e.g., by substitution or deletion of amino acid residues, such as substitution of glutamine for asparagine, or unglycosylated recombinant proteins may be produced by expressing the proteins in host cells that will not glycosylate them, e.g. in E. coli or glycosylation-deficient yeast. Examples of these approaches are described in more detail in WO 03/066824 and WO 01/79480, which are incorporated by reference, and are known in the art.

In various embodiments, the albumin fusion proteins of the invention are capable of a therapeutic activity and/or biologic activity corresponding to the therapeutic activity and/or biologic activity of the Therapeutic protein corresponding to the Therapeutic protein portion of the albumin fusion. In further embodiments, the therapeutically active protein portions of the albumin fusion proteins of the invention are fragments or variants of the reference sequence and are capable of the therapeutic activity and/or biologic activity of the corresponding Therapeutic protein.

### Polypeptide and Polynucleotide Fragments and Variants

### Fragments

The present invention is further directed to fragments of the Therapeutic proteins, albumin proteins, and/or albumin fusion proteins of the invention. Even if deletion of one or more amino acids from the N-terminus of a protein results in modification or loss of one or more biological functions of the Therapeutic protein, albumin protein, and/or albumin fusion protein, other Therapeutic activities and/or functional activities (e.g., biological activities, ability to multimerize, ability to bind a ligand) may still be retained. For example, the ability of polypeptides with N-terminal deletions to induce and/or bind to antibodies which recognize the complete or mature forms of the polypeptides generally will be retained when less than the majority of the residues of the complete polypeptide are removed from the N-terminus. Whether a particular polypeptide lacking N-terminal residues of a complete polypeptide retains such immunologic activities can readily be determined by routine methods described herein and otherwise known in the art. It is not unlikely that a mutein with a large number of deleted N-terminal amino acid residues may retain some biological or immunogenic activities. In fact, peptides composed of as few as six amino acid residues may often evoke an immune response.

Accordingly, fragments of a Therapeutic protein corresponding to a Therapeutic protein portion of an albumin fusion protein of the invention, include the full length protein as well as polypeptides having one or more residues deleted from the amino terminus of the amino acid sequence of the reference polypeptide. Polynucleotides encoding these polypeptides are also encompassed by the invention.

In addition, fragments of serum albumin polypeptides corresponding to an albumin protein portion of an albumin fusion protein of the invention, include the full length protein as well as polypeptides having one or more residues deleted from the amino terminus of the amino acid sequence of the reference polypeptide (i.e., serum albumin). Polynucleotides encoding these polypeptides are also encompassed by the invention.

Moreover, fragments of albumin fusion proteins of the invention include the full length albumin fusion protein as well as polypeptides having one or more residues deleted from the amino terminus of the albumin fusion protein. Polynucleotides encoding these polypeptides are also encompassed by the invention.

The present invention further provides polypeptides having one or more residues deleted from the carboxy terminus of the amino acid sequence of a Therapeutic protein corresponding to a Therapeutic protein portion of an albumin fusion protein of the invention. Polynucleotides encoding these polypeptides are also encompassed by the invention.

In addition, the present invention provides polypeptides having one or more residues deleted from the carboxy terminus of the amino acid sequence of an albumin protein corresponding to an albumin protein portion of an albumin fusion protein of the invention (e.g., serum albumin). Polynucleotides encoding these polypeptides are also encompassed by the invention.

Moreover, the present invention provides polypeptides having one or more residues deleted from the carboxy terminus of an albumin fusion protein of the invention. Polynucleotides encoding these polypeptides are also encompassed by the invention.

In addition, any of the above described N- or C-terminal deletions can be combined to produce a N- and C-terminal deleted reference polypeptide (e.g., a Therapeutic protein referred to in Table 1, or serum albumin (e.g., SEQ ID NO:18), or an albumin fusion protein of the invention). The invention also provides polypeptides having one or more amino acids deleted from both the amino and the carboxyl termini. Polynucleotides encoding these polypeptides are also encompassed by the invention.

The present application is also directed to proteins containing polypeptides at least 60%, 80%, 85%, 90%, 95%, 96%, 97%, 98% or 99% identical to a reference polypeptide sequence (e.g., a Therapeutic protein, serum albumin protein or an albumin fusion protein of the invention) set forth herein, or fragments thereof. In some embodiments, the application is directed to proteins comprising polypeptides at least 60%, 80%, 85%, 90%, 95%, 96%, 97%, 98% or 99% identical to reference polypeptides having the amino acid sequence of N- and C-terminal deletions as described above. Polynucleotides encoding these polypeptides are also encompassed by the invention.

Other polypeptide fragments of the invention are fragments comprising, or alternatively, consisting of, an amino acid sequence that displays a Therapeutic activity and/or functional activity (e.g. biological activity) of the polypeptide sequence of the Therapeutic protein or serum albumin protein of which the amino acid sequence is a fragment.

Other polypeptide fragments are biologically active fragments. Biologically active fragments are those exhibiting activity similar, but not necessarily identical, to an activity of the polypeptide of the present invention. The biological activity of the fragments may include an improved desired activity, or a decreased undesirable activity.

### Variants

“Variant” refers to a polynucleotide or nucleic acid differing from a reference nucleic acid or polypeptide, but retaining essential properties thereof. Generally, variants are overall closely similar, and, in many regions, identical to the reference nucleic acid or polypeptide.

As used herein, “variant” refers to a Therapeutic protein portion of an albumin fusion protein of the invention, an albumin portion of an albumin fusion protein of the invention, or an albumin fusion protein differing in sequence from a Therapeutic protein, albumin protein, and/or albumin fusion protein of the invention, respectively, but retaining at least one functional and/or therapeutic property thereof as described elsewhere herein or otherwise known in the art. Generally, variants are overall very similar, and, in many regions, identical to the amino acid sequence of the Therapeutic protein corresponding to a Therapeutic protein portion of an albumin fusion protein of the invention, albumin protein corresponding to an albumin protein portion of an albumin fusion protein of the invention, and/or albumin fusion protein of the invention. Nucleic acids encoding these variants are also encompassed by the invention.

The present invention is also directed to proteins which comprise, or alternatively consist of, an amino acid sequence which is at least 60%, 80%, 85%, 90%, 95%, 96%, 97%, 98%, 99% or 100%, identical to, for example, the amino acid sequence of a Therapeutic protein corresponding to a Therapeutic protein portion of an albumin fusion protein of the invention, albumin proteins (e.g., SEQ ID NO:18 or fragments or variants thereof) corresponding to an albumin protein portion of an albumin fusion protein of the invention, and/or albumin fusion proteins of the invention. Fragments of these polypeptides are also provided (e.g., those fragments described herein). Further polypeptides encompassed by the invention are polypeptides encoded by polynucleotides which hybridize to the complement of a nucleic acid molecule encoding an amino acid sequence of the invention under stringent hybridization conditions (e.g., hybridization to filter bound DNA in 6× Sodium chloride/Sodium citrate (SSC) at about 45 degrees Celsius, followed by one or more washes in 0.2×SSC, 0.1% SDS at about 50-65 degrees Celsius), under highly stringent conditions (e.g., hybridization to filter bound DNA in 6× sodium chloride/Sodium citrate (SSC) at about 45 degrees Celsius, followed by one or more washes in 0.1×SSC, 0.2% SDS at about 68 degrees Celsius), or under other stringent hybridization conditions which are known to those of skill in the art (see, for example, Ausubel, F. M. et al., eds., 1989 Current protocol in Molecular Biology, Green publishing associates, Inc., and John Wiley & Sons Inc., New York, at pages 6.3.1-6.3.6 and 2.10.3). Polynucleotides encoding these polypeptides are also encompassed by the invention.

By a polypeptide having an amino acid sequence at least, for example, 95% “identical” to a query amino acid sequence of the present invention, it is intended that the amino acid sequence of the subject polypeptide is identical to the query sequence except that the subject polypeptide sequence may include up to five amino acid alterations per each 100 amino acids of the query amino acid sequence. In other words, to obtain a polypeptide having an amino acid sequence at least 95% identical to a query amino acid sequence, up to 5% of the amino acid residues in the subject sequence may be inserted, deleted, or substituted with another amino acid. These alterations of the reference sequence may occur at the amino- or carboxy-terminal positions of the reference amino acid sequence or anywhere between those terminal positions, interspersed either individually among residues in the reference sequence or in one or more contiguous groups within the reference sequence.

As a practical matter, whether any particular polypeptide is at least 60%, 80%, 85%, 90%, 95%, 96%, 97%, 98% or 99% identical to, for instance, the amino acid sequence of an albumin fusion protein of the invention or a fragment thereof (such as the Therapeutic protein portion of the albumin fusion protein or the albumin portion of the albumin fusion protein), can be determined conventionally using known computer programs. Such programs and methods of using them are described, e.g., in. WO 03/066824 and WO 01/79480 (pp. 41-43), which are incorporated by reference herein.

The polynucleotide variants of the invention may contain alterations in the coding regions, non-coding regions, or both. Polynucleotide variants include those containing alterations which produce silent substitutions, additions, or deletions, but do not alter the properties or activities of the encoded polypeptide. Such nucleotide variants may be produced by silent substitutions due to the degeneracy of the genetic code. Polypeptide variants include those in which less than 50, less than 40, less than 30, less than 20, less than 10, or 5-50, 5-25, 5-10, 1-5, or 1-2 amino acids are substituted, deleted, or added in any combination. Polynucleotide variants can be produced for a variety of reasons, e.g., to optimize codon expression for a particular host (change codons in the human mRNA to those preferred by a microbial host, such as, yeast or E. coli).

In another embodiment, a polynucleotide encoding an albumin portion of an albumin fusion protein of the invention is optimized for expression in yeast or mammalian cells. In a further embodiment, a polynucleotide encoding a Therapeutic protein portion of an albumin fusion protein of the invention is optimized for expression in yeast or mammalian cells. In a still further embodiment, a polynucleotide encoding an albumin fusion protein of the invention is optimized for expression in yeast or mammalian cells.

In an alternative embodiment, a codon optimized polynucleotide encoding a Therapeutic protein portion of an albumin fusion protein of the invention does not hybridize to the wild type polynucleotide encoding the Therapeutic protein under stringent hybridization conditions as described herein. In a further embodiment, a codon optimized polynucleotide encoding an albumin portion of an albumin fusion protein of the invention does not hybridize to the wild type polynucleotide encoding the albumin protein under stringent hybridization conditions as described herein. In another embodiment, a codon optimized polynucleotide encoding an albumin fusion protein of the invention does not hybridize to the wild type polynucleotide encoding the Therapeutic protein portion or the albumin protein portion under stringent hybridization conditions as described herein.

In an additional embodiment, polynucleotides encoding a Therapeutic protein portion of an albumin fusion protein of the invention do not comprise, or alternatively consist of, the naturally occurring sequence of that Therapeutic protein. In a further embodiment, polynucleotides encoding an albumin protein portion of an albumin fusion protein of the invention do not comprise, or alternatively consist of, the naturally occurring sequence of albumin protein. In an alternative embodiment, polynucleotides encoding an albumin fusion protein of the invention do not comprise, or alternatively consist of, the naturally occurring sequence of a Therapeutic protein portion or the albumin protein portion.

In an additional embodiment, the Therapeutic protein may be selected from a random peptide library by biopanning, as there will be no naturally occurring wild type polynucleotide.

Naturally occurring variants are called “allelic variants,” and refer to one of several alternative forms of a gene occupying a given locus on a chromosome of an organism. (Genes II, Lewin, B., ed., John Wiley & Sons, New York (1985)). These allelic variants can vary at either the polynucleotide and/or polypeptide level and are included in the present invention. Alternatively, non-naturally occurring variants may be produced by mutagenesis techniques or by direct synthesis.

Using known methods of protein engineering and recombinant DNA technology, variants may be generated to improve or alter the characteristics of the polypeptides of the present invention. For instance, one or more amino acids may be deleted from the N-terminus or C-terminus of the polypeptide of the present invention without substantial loss of biological function. See, e.g., Ron et al., J. Biol. Chem. 268: 2984-2988 (1993) (KGF variants) and Dobeli et al., J. Biotechnology 7:199-216 (1988) (interferon gamma variants).

Moreover, ample evidence demonstrates that variants often retain a biological activity similar to that of the naturally occurring protein (e.g. Gayle and co-workers (J. Biol. Chem. 268:22105-22111 (1993) (IL-1a variants)). Furthermore, even if deleting one or more amino acids from the N-terminus or C-terminus of a polypeptide results in modification or loss of one or more biological functions, other biological activities may still be retained. For example, the ability of a deletion variant to induce and/or to bind antibodies which recognize the secreted form will likely be retained when less than the majority of the residues of the secreted form are removed from the N-terminus or C-terminus. Whether a particular polypeptide lacking N- or C-terminal residues of a protein retains such immunogenic activities can readily be determined by routine methods described herein and otherwise known in the art.

Thus, the invention further includes polypeptide variants which have a functional activity (e.g., biological activity and/or therapeutic activity). In further embodiments the invention provides variants of albumin fusion proteins that have a functional activity (e.g., biological activity and/or therapeutic activity, such as that disclosed in the “Biological Activity” column in Table 1) that corresponds to one or more biological and/or therapeutic activities of the Therapeutic protein corresponding to the Therapeutic protein portion of the albumin fusion protein. Such variants include deletions, insertions, inversions, repeats, and substitutions selected according to general rules known in the art so as have little effect on activity.

In other embodiments, the variants of the invention have conservative substitutions. By “conservative substitutions” is intended swaps within groups such as replacement of the aliphatic or hydrophobic amino acids Ala, Val, Leu and Ile; replacement of the hydroxyl residues Ser and Thr; replacement of the acidic residues Asp and Glu; replacement of the amide residues Asn and Gln, replacement of the basic residues Lys, Arg, and His; replacement of the aromatic residues Phe, Tyr, and Trp, and replacement of the small-sized amino acids Ala, Ser, Thr, Met, and Gly.

Guidance concerning how to make phenotypically silent amino acid substitutions is provided, for example, in Bowie et al., “Deciphering the Message in Protein Sequences: Tolerance to Amino Acid Substitutions,” Science 247:1306-1310 (1990), wherein the authors indicate that there are two main strategies for studying the tolerance of an amino acid sequence to change.

As the authors state, proteins are surprisingly tolerant of amino acid substitutions. The authors further indicate which amino acid changes are likely to be permissive at certain amino acid positions in the protein. For example, most buried (within the tertiary structure of the protein) amino acid residues require nonpolar side chains, whereas few features of surface side chains are generally conserved. Moreover, tolerated conservative amino acid substitutions involve replacement of the aliphatic or hydrophobic amino acids Ala, Val, Leu and Ile; replacement of the hydroxyl residues Ser and Thr; replacement of the acidic residues Asp and Glu; replacement of the amide residues Asn and Gln, replacement of the basic residues Lys, Arg, and His; replacement of the aromatic residues Phe, Tyr, and Trp, and replacement of the small-sized amino acids Ala, Ser, Thr, Met, and Gly.

Besides conservative amino acid substitution, variants of the present invention include (i) polypeptides containing substitutions of one or more of the non-conserved amino acid residues, where the substituted amino acid residues may or may not be one encoded by the genetic code, or (ii) polypeptides containing substitutions of one or more of the amino acid residues having a substituent group, or (iii) polypeptides which have been fused with or chemically conjugated to another compound, such as a compound to increase the stability and/or solubility of the polypeptide (for example, polyethylene glycol), (iv) polypeptide containing additional amino acids, such as, for example, an IgG Fc fusion region peptide. Such variant polypeptides are deemed to be within the scope of those skilled in the art from the teachings herein.

For example, polypeptide variants containing amino acid substitutions of charged amino acids with other charged or neutral amino acids may produce proteins with improved characteristics, such as less aggregation. Aggregation of pharmaceutical formulations both reduces activity and increases clearance due to the aggregate's immunogenic activity. See Pinckard et al., Clin. Exp. Immunol. 2:331-340 (1967); Robbins et al, Diabetes 36: 838-845 (1987); Cleland et al., Crit. Rev. Therapeutic Drug Carrier Systems 10:307-377 (1993).

In specific embodiments, the polypeptides of the invention comprise, or alternatively, consist of, fragments or variants of the amino acid sequence of a Therapeutic protein described herein and/or human serum albumin, and/or albumin fusion protein of the invention, wherein the fragments or variants have 1-5,5-10, 5-25, 5-50, 10-50 or 50-150, amino acid residue additions, substitutions, and/or deletions when compared to the reference amino acid sequence. In certain embodiments, the amino acid substitutions are conservative. Nucleic acids encoding these polypeptides are also encompassed by the invention.

The polypeptide of the present invention can be composed of amino acids joined to each other by peptide bonds or modified peptide bonds, i.e., peptide isosteres, and may contain amino acids other than the 20 gene-encoded amino acids. The polypeptides may be modified by either natural processes, such as post-translational processing, or by chemical modification techniques which are well known in the art. Such modifications are well described in basic texts and in more detailed monographs, as well as in a voluminous research literature. Modifications can occur anywhere in a polypeptide, including the peptide backbone, the amino acid side-chains and the amino or carboxyl termini. It will be appreciated that the same type of modification may be present in the same or varying degrees at several sites in a given polypeptide. Also, a given polypeptide may contain many types of modifications. Polypeptides may be branched, for example, as a result of ubiquitination, and they may be cyclic, with or without branching. Cyclic, branched, and branched cyclic polypeptides may result from posttranslational natural processes or may be made by synthetic methods. Modifications include acetylation, acylation, ADP-ribosylation, amidation, covalent attachment of flavin, covalent attachment of a heme moiety, covalent attachment of a nucleotide or nucleotide derivative, covalent attachment of a lipid or lipid derivative, covalent attachment of phosphatidylinositol, cross-linking, cyclization, disulfide bond formation, demethylation, formation of covalent cross-links, formation of cysteine, formation of pyroglutamate, formylation, gamma-carboxylation, glycosylation, GPI anchor formation, hydroxylation, iodination, methylation, myristylation, oxidation, pegylation, proteolytic processing, phosphorylation, prenylation, racemization, selenoylation, sulfation, transfer-RNA mediated addition of amino acids to proteins such as arginylation, and ubiquitination.

Furthermore, chemical entities may be covalently attached to the albumin fusion proteins to enhance or modulate a specific functional or biological activity such as by methods disclosed in Current Opinions in Biotechnology, 10:324 (1999).

Additional post-translational modifications encompassed by the invention include, for example, e.g., N-linked or O-linked carbohydrate chains, processing of N-terminal or C-terminal ends), attachment of chemical moieties to the amino acid backbone, chemical modifications of N-linked or O-linked carbohydrate chains, and addition or deletion of an N-terminal methionine residue as a result of prokaryotic host cell expression. The albumin fusion proteins may also be modified with, e.g., but not limited to, a chemotherapeutic agent, such as a drug, and/or a detectable label, such as an enzymatic, fluorescent, isotopic and/or affinity label to allow for detection and isolation of the protein. Examples of such modifications are given, e.g., in WO 03/066824 and in WO 01/79480 (pp. 105-106), which are incorporated by reference herein.

### Functional Activity

“A polypeptide having functional activity” refers to a polypeptide capable of displaying one or more known functional activities associated with the full-length, pro-protein, and/or mature form of a Therapeutic protein. Such functional activities include, but are not limited to, biological activity, antigenicity (ability to bind (or compete with a polypeptide for binding) to an anti-polypeptide antibody), immunogenicity (ability to generate antibody which binds to a specific polypeptide of the invention), ability to form multimers with polypeptides of the invention, and ability to bind to a receptor or ligand for a polypeptide.

“A polypeptide having biological activity” refers to a polypeptide exhibiting activity similar to, but not necessarily identical to, an activity of a Therapeutic protein of the present invention, including mature forms, as measured in a particular biological assay, with or without dose dependency. In the case where dose dependency does exist, it need not be identical to that of the polypeptide, but rather substantially similar to the dose-dependence in a given activity as compared to the polypeptide of the present invention.

In other embodiments, an albumin fusion protein of the invention has at least one biological and/or therapeutic activity associated with the Therapeutic protein (or fragment or variant thereof) when it is not fused to albumin.

The albumin fusion proteins of the invention can be assayed for functional activity (e.g., biological activity) using or routinely modifying assays known in the art, as well as assays described herein. Specifically, albumin fusion proteins may be assayed for functional activity. Additionally, one of skill in the art may routinely assay fragments of a Therapeutic protein corresponding to a Therapeutic protein portion of an albumin fusion protein of the invention, for activity using assays for IL-11 activity. Further, one of skill in the art may routinely assay fragments of an albumin protein corresponding to an albumin protein portion of an albumin fusion protein of the invention, for activity using assays known in the art and/or as described in the Examples section in WO 03/066824 and WO 01/79480.

In addition, assays described herein (see Examples and Table 1) and otherwise known in the art may routinely be applied to measure the ability of albumin fusion proteins of the present invention and fragments, variants and derivatives thereof to elicit biological activity and/or Therapeutic activity (either in vitro or in vivo) related to either the Therapeutic protein portion and/or albumin portion of the albumin fusion protein of the present invention. Other methods will be known to the skilled artisan and are within the scope of the invention.

### Expression of Fusion Proteins

The albumin fusion proteins of the invention may be produced as recombinant molecules by secretion from yeast, a microorganism such as a bacterium, or a human or animal cell line. Optionally, the polypeptide is secreted from the host cells.

For expression of the albumin fusion proteins exemplified herein, yeast strains disrupted in the HSP150 gene as exemplified in WO 95/33833, or yeast strains disrupted in the PMT1 gene as exemplified in WO 00/44772 (serving to reduce/eliminate O-linked glycosylation of the albumin fusions), or yeast strains disrupted in the YAP3 gene as exemplified in WO 95/23857 were successfully used, in combination with the yeast PRB1 promoter, the HSA/MFα-1 fusion leader sequence exemplified in WO 90/01063, the yeast ADH1 terminator, the LEU2 selection marker and the disintegration vector pSAC35 exemplified in U.S. Pat. No. 5,637,504.

Other yeast strains, promoters, leader sequences, terminators, markers and vectors which are expected to be useful in the invention are described in WO 03/066824 and in WO 01/74980 (pp. 94-99), which are incorporated herein by reference.

The present invention also includes a cell, optionally a yeast cell transformed to express an albumin fusion protein of the invention. In addition to the transformed host cells themselves, the present invention also contemplates a culture of those cells, optionally a monoclonal (clonally homogeneous) culture, or a culture derived from a monoclonal culture, in a nutrient medium. If the polypeptide is secreted, the medium will contain the polypeptide, with the cells, or without the cells if they have been filtered or centrifuged away. Many expression systems are known and may be used, including bacteria (for example E. coli and Bacillus subtilis), yeasts (for example Saccharomyces cerevisiae, Kluyveromyces lactis and Pichia pastoris), filamentous fungi (for example Aspergillus), plant cells, animal cells and insect cells.

The desired protein is produced in conventional ways, for example from a coding sequence inserted in the host chromosome or on a free plasmid. The yeasts are transformed with a coding sequence for the desired protein in any of the usual ways, for example electroporation. Methods for transformation of yeast by electroporation are disclosed in Becker & Guarente (1990) Methods Enzymol. 194, 182.

Successfully transformed cells, i.e., cells that contain a DNA construct of the present invention, can be identified by well known techniques. For example, cells resulting from the introduction of an expression construct can be grown to produce the desired polypeptide. Cells can be harvested and lysed and their DNA content examined for the presence of the DNA using a method such as that described by Southern (1975) J. Mol. Biol. 98, 503 or Berent et al. (1985) Biotech. 3, 208. Alternatively, the presence of the protein in the supernatant can be detected using antibodies.

Useful yeast plasmid vectors include pRS403-406 and pRS413-416 and are generally available from Stratagene Cloning Systems, La Jolla, Calif. 92037, USA. Plasmids pRS403, pRS404, pRS405 and pRS406 are Yeast Integrating plasmids (YIps) and incorporate the yeast selectable markers HIS3, TRP1, LEU2 and UR43. Plasmids pRS413-416 are Yeast Centromere plasmids (YCps).

Vectors for making albumin fusion proteins for expression in yeast include pPPC0005, pScCHSA, pScNHSA, and pC4:HSA which were deposited on Apr. 11, 2001 at the American Type Culture Collection, 10801 University Boulevard, Manassas, Va. 20110-2209 and which are described in WO 03/066824 and WO 01/79480, which are incorporated by reference herein.

Another vector which is expected to be useful for expressing an albumin fusion protein in yeast is the pSAC35 vector which is described in Sleep et al., BioTechnology 8:42 (1990), which is hereby incorporated by reference in its entirety. The plasmid pSAC35 is of the disintegration class of vector described in U.S. Pat. No. 5,637,504.

A variety of methods have been developed to operably link DNA to vectors via complementary cohesive termini. For instance, complementary homopolymer tracts can be added to the DNA segment to be inserted to the vector DNA. The vector and DNA segment are then joined by hydrogen bonding between the complementary homopolymeric tails to form recombinant DNA molecules.

Synthetic linkers containing one or more restriction sites provide an alternative method of joining the DNA segment to vectors. The DNA segment, generated by endonuclease restriction digestion, is treated with bacteriophage T4 DNA polymerase or E. coli DNA polymerase I, which are enzymes that remove protruding, 5′-single-stranded termini with their 3′-5′-exonucleolytic activities and fill in recessed 3′-ends with their polymerizing activities. The combination of these activities therefore generates blunt-ended DNA segments. The blunt-ended segments are then incubated with a large molar excess of linker molecules in the presence of an enzyme that is able to catalyze the ligation of blunt-ended DNA molecules, such as bacteriophage T4 DNA ligase. Thus, the products of the reaction are DNA segments carrying polymeric linker sequences at their ends. These DNA segments are then cleaved with the appropriate restriction enzyme and ligated to an expression vector that has been cleaved with an enzyme that produces termini compatible with those of the DNA segment.

Synthetic linkers containing a variety of restriction endonuclease sites are commercially available from a number of commercial sources.

A desirable way to modify the DNA in accordance with the invention, if, for example, HA variants are to be prepared, is to use the polymerase chain reaction as disclosed by Saiki et al. (1988) Science 239, 487-491. In this method the DNA to be enzymatically amplified is flanked by two specific oligonucleotide primers which themselves become incorporated into the amplified DNA. The specific primers may contain restriction endonuclease recognition sites which can be used for cloning into expression vectors using methods known in the art.

Exemplary genera of yeast contemplated to be useful in the practice of the present invention as hosts for expressing the albumin fusion proteins are Pichia (formerly classified as Hansenula), Saccharomyces, Kluyveromyces, Aspergillus, Candida Torulopsis, Torulaspora, Schizosaccharonyces, Citeromyces, Pachysolen, Zygosaccharomyces, Debaromyces, Trichoderma, Cephalosporium, Humicola, Mucor, Neurospora, Yarrowia, Metschunikowia, Rhodosporidium, Leucosporidium, Botryoascus, Sporidiobolus, Endomycopsis, and the like. Genera include those selected from the group consisting of Saccharomyces, Schizosaccharomyces, Kluyveromyces, Pichia and Torulaspora. Examples of Saccharomyces spp. are S. cerevisiae, S. italicus and S. rouxii. Examples of other species, and methods of transforming them, are described in WO 03/066824 and WO 01/79480 (pp. 97-98), which are incorporated herein by reference.

Methods for the transformation of S. cerevisiae are taught generally in EP 251 744, EP 258 067 and WO 90/01063, all of which are incorporated herein by reference. Suitable promoters for S. cerevisiae include those associated with the PGKI gene, GAL1 or GAL10 genes, CYCI, PHO5, TRPI, ADHI, ADH2, the genes for glyceraldehyde-3-phosphate dehydrogenase, hexokinase, pyruvate decarboxylase, phosphofructokinase, triose phosphate isomerase, phosphoglucose isomerase, glucokinase, alpha-mating factor pheromone (a mating factor pheromone), the PRB1 promoter, the GUT2 promoter, the GPD1 promoter, and hybrid promoters involving hybrids of parts of 5′ regulatory regions with parts of 5′ regulatory regions of other promoters or with upstream activation sites (e.g. the promoter of EP-A-258 067).

Convenient regulatable promoters for use in Schizosaccharomyces pombe are the thiamine-repressible promoter from the nmt gene as described by Maundrell (1990) J. Biol. Chem. 265, 10857-10864 and the glucose repressible jbp1 gene promoter as described by Hoffman & Winston (1990) Genetics 124, 807-816.

Methods of transforming Pichia for expression of foreign genes are taught in, for example, Cregg et al. (1993), and various Phillips patents (e.g. U.S. Pat. No. 4,857,467, incorporated herein by reference), and Pichia expression kits are commercially available from Invitrogen BV, Leek, Netherlands, and Invitrogen Corp., San Diego, Calif. Suitable promoters include AOXI and AOX2. Gleeson et al (1986) J. Gen. Microbiol. 132, 3459-3465 include information on Hansenula vectors and transformation, suitable promoters being MOX1 and FMD1; whilst EP 361 991, Fleer et al. (1991) and other publications from Rhone-Poulenc Rorer teach how to express foreign proteins in Kluyveromyces spp.

The transcription termination signal may be the 3′ flanking sequence of a eukaryotic gene which contains proper signals for transcription termination and polyadenylation. Suitable 3′ flanking sequences may, for example, be those of the gene naturally linked to the expression control sequence used, i.e. may correspond to the promoter. Alternatively, they may be different in which case the termination signal of the S. cerevisiae ADHI gene is optionally used.

The desired albumin fusion protein may be initially expressed with a secretion leader sequence, which may be any leader effective in the yeast chosen. Leaders useful in S. cerevisiae include that from the mating factor polypeptide (MF_{α1}) and the hybrid leaders of EP-A-387 319. Such leaders (or signals) are cleaved by the yeast before the mature albumin is released into the surrounding medium. Further such leaders include those of S. cerevisiae invertase (SUC2) disclosed in JP 62-096086 (granted as 911036516), acid phosphatase (PH05), the pre-sequence of MFα-1, β-glucanase (BGL2) and killer toxin; S. diastaticus glucoamylase II; S. carlsbergensis α-galactosidase (MEL1); K. lactis killer toxin; and Candida glucoamylase.

### Additional Methods of Recombinant and Synthetic Production of Albumin Fusion Proteins

The present invention includes polynucleotides encoding albumin fusion proteins of this invention, as well as vectors, host cells and organisms containing these polynucleotides. The present invention also includes methods of producing albumin fusion proteins of the invention by synthetic and recombinant techniques. The polynucleotides, vectors, host cells, and organisms may be isolated and purified by methods known in the art

A vector useful in the invention may be, for example, a phage, plasmid, cosmid, mini-chromosome, viral or retroviral vector. The vectors which can be utilized to clone and/or express polynucleotides of the invention are vectors which are capable of replicating and/or expressing the polynucleotides in the host cell in which the polynucleotides are desired to be replicated and/or expressed. In general, the polynucleotides and/or vectors can be utilized in any cell, either eukaryotic or prokaryotic, including mammalian cells (e.g., human (e.g., HeLa), monkey (e.g., Cos), rabbit (e.g., rabbit reticulocytes), rat, hamster (e.g., CHO, NSO and baby hamster kidney cells) or mouse cells (e.g., L cells), plant cells, yeast cells, insect cells or bacterial cells (e.g., E. coli). See, e.g., F. Ausubel et al., Current Protocols in Molecular Biology, Greene Publishing Associates and Wiley-Interscience (1992) and Sambrook et al. (1989) for examples of appropriate vectors for various types of host cells. Note, however, that when a retroviral vector that is replication defective is used, viral propagation generally will occur only in complementing host cells.

The host cells containing these polynucleotides can be used to express large amounts of the protein useful in, for example, pharmaceuticals, diagnostic reagents, vaccines and therapeutics. The protein may be isolated and purified by methods known in the art or described herein.

The polynucleotides encoding albumin fusion proteins of the invention may be joined to a vector containing a selectable marker for propagation in a host. Generally, a plasmid vector may be introduced in a precipitate, such as a calcium phosphate precipitate, or in a complex with a charged lipid. If the vector is a virus, it may be packaged in vitro using an appropriate packaging cell line and then transduced into host cells.

The polynucleotide insert should be operatively linked to an appropriate promoter compatible with the host cell in which the polynucleotide is to be expressed. The promoter may be a strong promoter and/or an inducible promoter. Examples of promoters include the phage lambda PL promoter, the E. coli lac, trp, phoA and tac promoters, the SV40 early and late promoters and promoters of retroviral LTRs, to name a few. Other suitable promoters will be known to the skilled artisan. The expression constructs will further contain sites for transcription initiation, termination, and, in the transcribed region, a ribosome binding site for translation. The coding portion of the transcripts expressed by the constructs may include a translation initiating codon at the beginning and a termination codon (UAA, UGA or UAG) appropriately positioned at the end of the polypeptide to be translated.

As indicated, the expression vectors may include at least one selectable marker. Such markers include dihydrofolate reductase, G418, glutamine synthase, or neomycin resistance for eukaryotic cell culture, and tetracycline, kanamycin or ampicillin resistance genes for culturing in E. coli and other bacteria. Representative examples of appropriate hosts include, but are not limited to, bacterial cells, such as E. coli, Streptomyces and Salmonella typhimurium cells; fungal cells, such as yeast cells (e.g., Saccharomyces cerevisiae or Pichia pastoris (ATCC Accession No. 201178)); insect cells such as Drosophila S2 and Spodoptera Sf9 cells; animal cells such as CHO, COS, NSO, 293, and Bowes melanoma cells; and plant cells. Appropriate culture mediums and conditions for the above-described host cells are known in the art.

In one embodiment, polynucleotides encoding an albumin fusion protein of the invention may be fused to signal sequences which will direct the localization of a protein of the invention to particular compartments of a prokaryotic or eukaryotic cell and/or direct the secretion of a protein of the invention from a prokaryotic or eukaryotic cell. For example, in E. coli, one may wish to direct the expression of the protein to the periplasmic space. Examples of signal sequences or proteins (or fragments thereof) to which the albumin fusion proteins of the invention may be fused in order to direct the expression of the polypeptide to the periplasmic space of bacteria include, but are not limited to, the pelB signal sequence, the maltose binding protein (MBP) signal sequence, MBP, the ompA signal sequence, the signal sequence of the periplasmic E. coli heat-labile enterotoxin B-subunit, and the signal sequence of alkaline phosphatase. Several vectors are commercially available for the construction of fusion proteins which will direct the localization of a protein, such as the pMAL series of vectors (particularly the pMAL-p series) available from New England Biolabs. In a specific embodiment, polynucleotides albumin fusion proteins of the invention may be fused to the pelB pectate lyase signal sequence to increase the efficiency of expression and purification of such polypeptides in Gram-negative bacteria. See, U.S. Pat. Nos. 5,576,195 and 5,846,818, the contents of which are herein incorporated by reference in their entireties.

Examples of signal peptides that may be fused to an albumin fusion protein of the invention in order to direct its secretion in mammalian cells include, but are not limited to, the MPIF-1 signal sequence (e.g., amino acids 1-21 of GenBank Accession number AAB51134), the stanniocalcin signal sequence (MLQNSAVLLLLVISASA, SEQ ID NO:10) and a consensus signal sequence (MPTWAWWLFLVLLLALWAPARG, SEQ ID NO:11). A suitable signal sequence that may be used in conjunction with baculoviral expression systems is the gp67 signal sequence (e.g., amino acids 1-19 of GenBank Accession Number AAA72759).

Vectors which use glutamine synthase (GS) or DHFR as the selectable markers can be amplified in the presence of the drugs methionine sulphoximine or methotrexate, respectively. An advantage of glutamine synthase based vectors are the availability of cell lines (e.g., the murine myeloma cell line, NSO) which are glutamine synthase negative. Glutamine synthase expression systems can also function in glutamine synthase expressing cells (e.g., Chinese Hamster Ovary (CHO) cells) by providing additional inhibitor to prevent the functioning of the endogenous gene. A glutamine synthase expression system and components thereof are detailed in PCT publications: WO7/04462; WO86/05807; WO89/01036; WO89/10404; and WO91/06657, which are hereby incorporated in their entireties by reference herein. Additionally, glutamine synthase expression vectors can be obtained from Lonza Biologics, Inc. (Portsmouth, N.H.). Expression and production of monoclonal antibodies using a GS expression system in murine myeloma cells is described in Bebbington et al., Bio/technology 10:169 (1992) and in Biblia & Robinson Biotechnol. Prog. 11:1 (1995) which are herein incorporated by reference.

The present invention also relates to host cells containing vector constructs, such as those described herein, and additionally encompasses host cells containing nucleotide sequences of the invention that are operably associated with one or more heterologous control regions (e.g., promoter and/or enhancer) using techniques known of in the art. The host cell can be a higher eucaryotic cell, such as a mammalian cell (e.g., a human derived cell), or a lower eukaryotic cell, such as a yeast cell, or the host cell can be a prokaryotic cell, such as a bacterial cell. A host strain may be chosen which modulates the expression of the inserted gene sequences, or modifies and processes the gene product in the specific fashion desired. Expression from certain promoters can be elevated in the presence of certain inducers; thus expression of the genetically engineered polypeptide may be controlled. Furthermore, different host cells have characteristics and specific mechanisms for the translational and post-translational processing and modification (e.g., phosphorylation, cleavage) of proteins. Appropriate cell lines can be chosen to ensure the desired modifications and processing of the foreign protein expressed.

Introduction of the nucleic acids and nucleic acid constructs of the invention into the host cell can be effected by calcium phosphate transfection, DEAE-dextran mediated transfection, cationic lipid-mediated transfection, electroporation, transduction, infection, or other methods. Such methods are described in many standard laboratory manuals, such as Davis et al., Basic Methods In Molecular Biology (1986). It is specifically contemplated that the polypeptides of the present invention may in fact be expressed by a host cell lacking a recombinant vector.

In addition to encompassing host cells containing the vector constructs discussed herein, the invention also encompasses primary, secondary, and immortalized host cells of vertebrate origin, particularly mammalian origin, that have been engineered to delete or replace endogenous genetic material (e.g., the coding sequence corresponding to a Therapeutic protein may be replaced with an albumin fusion protein corresponding to the Therapeutic protein), and/or to include genetic material (e.g., heterologous polynucleotide sequences such as for example, an albumin fusion protein of the invention corresponding to the Therapeutic protein may be included). The genetic material operably associated with the endogenous polynucleotide may activate, alter, and/or amplify endogenous polynucleotides.

In addition, techniques known in the art may be used to operably associate heterologous polynucleotides (e.g., polynucleotides encoding an albumin protein, or a fragment or variant thereof) and/or heterologous control regions (e.g., promoter and/or enhancer) with endogenous polynucleotide sequences encoding a Therapeutic protein via homologous recombination (see, e.g., U.S. Pat. No. 5,641,670; WO 96/29411; WO 94/12650; Koller et al., Proc. Natl. Acad. Sci. USA 86:8932-8935 (1989); and Zijlstra et al., Nature 342:435-438 (1989), the disclosures of each of which are incorporated by reference in their entireties).

Advantageously, albumin fusion proteins of the invention can be recovered and purified from recombinant cell cultures by well-known methods including ammonium sulfate or ethanol precipitation, acid extraction, anion or cation exchange chromatography, phosphocellulose chromatography, hydrophobic interaction chromatography, affinity chromatography, hydroxyapatite chromatography, hydrophobic charge interaction chromatography and lectin chromatography. In some embodiments, high performance liquid chromatography (“HPLC”) may be employed for purification.

In preferred some embodiments albumin fusion proteins of the invention are purified using one or more chromatography methods listed above. In other embodiments, albumin fusion proteins of the invention are purified using one or more of the following chromatography columns, Q Sepharose FF column, SP Sepharose FF column, Q Sepharose High Performance Column, Blue Sepharose FF column, Blue Column, Phenyl Sepharose FF column, DEAE Sepharose FF, or Methyl Column. “Sepharose” is a trademark.

Additionally, albumin fusion proteins of the invention may be purified using the process described in WO 00/44772 which is herein incorporated by reference in its entirety. One of skill in the art could easily modify the process described therein for use in the purification of albumin fusion proteins of the invention.

Albumin fusion proteins of the present invention may be recovered from: products produced by recombinant techniques from a prokaryotic or eukaryotic host, including, for example, bacterial, yeast, higher plant, insect, and mammalian cells. Depending upon the host employed in a recombinant production procedure, the polypeptides of the present invention may be glycosylated or may be non-glycosylated. In addition, albumin fusion proteins of the invention may also include an initial modified methionine residue, in some cases as a result of host-mediated processes. Thus, it is well known in the art that the N-terminal methionine encoded by the translation initiation codon generally is removed with high efficiency from any protein after translation in all eukaryotic cells. While the N-terminal methionine on most proteins also is efficiently removed in most prokaryotes, for some proteins, this prokaryotic removal process is inefficient, depending on the nature of the amino acid to which the N-terminal methionine is covalently linked.

Albumin fusion proteins of the invention and antibodies that bind a Therapeutic protein or fragments or variants thereof can be fused to marker sequences, such as a peptide to facilitate purification. In one embodiment, the marker amino acid sequence is a hexa-histidine peptide, such as the tag provided in a pQE vector (QIAGEN, Inc., 9259 Eton Avenue, Chatsworth, Calif., 91311), among others, many of which are commercially available. As described in Gentz et al., Proc. Natl. Acad. Sci. USA 86:821-824 (1989), for instance, hexa-histidine provides for convenient purification of the fusion protein. Other peptide tags useful for purification include, but are not limited to, the “HA” tag, which corresponds to an epitope derived from the influenza hemagglutinin protein (Wilson et al., Cell 37:767 (1984)) and the “FLAG” tag.

Further, an albumin fusion protein of the invention may be conjugated to a therapeutic moiety such as a cytotoxin, e.g., a cytostatic or cytocidal agent, a therapeutic agent or a radioactive metal ion, e.g., alpha-emitters such as, for example, 213Bi. Examples of such agents are given in WO 03/066824 and in WO 01/79480 (p. 107), which are incorporated herein by reference.

Albumin fusion proteins may also be attached to solid supports, which are particularly useful for immunoassays or purification of polypeptides that are bound by, that bind to, or associate with albumin fusion proteins of the invention. Such solid supports include, but are not limited to, glass, cellulose, polyacrylamide, nylon, polystyrene, polyvinyl chloride or polypropylene.

Also provided by the invention are chemically modified derivatives of the albumin fusion proteins of the invention which may provide additional advantages such as increased solubility, stability and circulating time of the polypeptide, or decreased immunogenicity (see U.S. Pat. No. 4,179,337). Examples involving the use of polyethylene glycol are given in WO 01/79480 (pp. 109-111), which are incorporated by reference herein.

The presence and quantity of albumin fusion proteins of the invention may be determined using ELISA, a well known immunoassay known in the art.

### Uses of the Polypeptides

Each of the polypeptides identified herein can be used in numerous ways. The following description should be considered exemplary and utilizes known techniques.

The albumin fusion proteins of the present invention are useful for treatment, prevention and/or prognosis of various disorders in mammals, preferably humans. Such disorders include, but are not limited to thrombocytopenia, vWD and inflammatory diseases, such as IBD.

Moreover, albumin fusion proteins of the present invention can be used to treat or prevent diseases or conditions. In addition, the albumin fusion proteins of the invention may be used as a prophylactic measure Albumin fusion proteins can be used to assay levels of polypeptides in a biological sample. Albumin fusion proteins of the invention can also be used to raise antibodies, which in turn may be used to measure protein expression of the Therapeutic protein, albumin protein, and/or the albumin fusion protein of the invention from a recombinant cell, as a way of assessing transformation of the host cell, or in a biological sample. Moreover, the albumin fusion proteins of the present invention can be used to test the biological activities described herein.

### Transgenic Organisms

Transgenic organisms that express the albumin fusion proteins of the invention are also included in the invention. Transgenic organisms are genetically modified organisms into which recombinant, exogenous or cloned genetic material has been transferred. Such genetic material is often referred to as a transgene. The nucleic acid sequence of the transgene may include one or more transcriptional regulatory sequences and other nucleic acid sequences such as introns, that may be necessary for optimal expression and secretion of the encoded protein. The transgene may be designed to direct the expression of the encoded protein in a manner that facilitates its recovery from the organism or from a product produced by the organism, e.g. from the milk, blood, urine, eggs, hair or seeds of the organism. The transgene may consist of nucleic acid sequences derived from the genome of the same species or of a different species than the species of the target animal. The transgene may be integrated either at a locus of a genome where that particular nucleic acid sequence is not otherwise normally found or at the normal locus for the transgene.

The term “germ cell line transgenic organism” refers to a transgenic organism in which the genetic alteration or genetic information was introduced into a germ line cell, thereby conferring the ability of the transgenic organism to transfer the genetic information to offspring. If such offspring in fact possess some or all of that alteration or genetic information, then they too are transgenic organisms. The alteration or genetic information may be foreign to the species of organism to which the recipient belongs, foreign only to the particular individual recipient, or may be genetic information already possessed by the recipient. In the last case, the altered or introduced gene may be expressed differently than the native gene.

A transgenic organism may be a transgenic human, animal or plant. Transgenics can be produced by a variety of different methods including transfection, electroporation, microinjection, gene targeting in embryonic stem cells and recombinant viral and retroviral infection (see, e.g., U.S. Pat. No. 4,736,866; U.S. Pat. No. 5,602,307; Mullins et al. (1993) Hypertension 22(4):630-633; Brenin et al. (1997) Surg. Oncol. 6(2) 99-110; Tuan (ed.), Recombinant Gene Expression Protocols, Methods in Molecular Biology No. 62, Humana Press (1997)). The method of introduction of nucleic acid fragments into recombination competent mammalian cells can be by any method which favours co-transformation of multiple nucleic acid molecules. Detailed procedures for producing transgenic animals are readily available to one skilled in the art, including the disclosures in U.S. Pat. No. 5,489,743 and U.S. Pat. No. 5,602,307. Additional information is given in WO 03/066824 and WO 01/79480 (pp. 151-162), which are incorporated by reference herein.

### Gene Therapy

Constructs encoding albumin fusion proteins of the invention can be used as a part of a gene therapy protocol to deliver therapeutically effective doses of the albumin fusion protein. One approach for in vivo introduction of nucleic acid into a cell is by use of a viral vector containing nucleic acid, encoding an albumin fusion protein of the invention. Infection of cells with a viral vector has the advantage that a large proportion of the targeted cells can receive the nucleic acid. Additionally, molecules encoded within the viral vector, e.g., by a cDNA contained in the viral vector, are expressed efficiently in cells which have taken up viral vector nucleic acid. The extended plasma half-life of the described albumin fusion proteins might even compensate for a potentially low expression level.

Retrovirus vectors and adeno-associated virus vectors can be used as a recombinant gene delivery system for the transfer of exogenous nucleic acid molecules encoding albumin fusion proteins in vivo. These vectors provide efficient delivery of nucleic acids into cells, and the transferred nucleic acids are stably integrated into the chromosomal DNA of the host. Examples of such vectors, methods of using them, and their advantages, as well as non-viral delivery methods are described in detail in WO 03/066824 and WO 01/79480 (pp. 151-153), which are incorporated by reference herein.

Gene delivery systems for a gene encoding an albumin fusion protein of the invention can be introduced into a patient by any of a number of methods. For instance, a pharmaceutical preparation of the gene delivery system can be introduced systemically, e.g. by intravenous injection, and specific transduction of the protein in the target cells occurs predominantly from specificity of transfection provided by the gene delivery vehicle, cell-type or tissue-type expression due to the transcriptional regulatory sequences controlling expression of the receptor gene, or a combination thereof. In other embodiments, initial delivery of the recombinant gene is more limited with introduction into the animal being quite localized. For example, the gene delivery vehicle can be introduced by catheter (see U.S. Pat. No. 5,328,470) or by stereotactic injection (e.g. Chen et al. (1994) PNAS 91: 3054-3057). The pharmaceutical preparation of the gene therapy construct can consist essentially of the gene delivery system in an acceptable diluent, or can comprise a slow release matrix in which the gene delivery vehicle is imbedded. Where the albumin fusion protein can be produced intact from recombinant cells, e.g. retroviral vectors, the pharmaceutical preparation can comprise one or more cells which produce the albumin fusion protein. Additional gene therapy methods are described in WO 03/066824 and in WO 01/79480 (pp. 153-162), which are incorporated herein by reference.

### Pharmaceutical or Therapeutic Compositions

The albumin fusion proteins of the invention or formulations thereof may be administered by any conventional method including parenteral (e.g. subcutaneous or intramuscular) injection or intravenous infusion. The treatment may consist of a single dose or a plurality of doses over a period of time. Furthermore, the dose, or plurality of doses, is administered less frequently than for the Therapeutic Protein which is not fused to albumin.

While it is possible for an albumin fusion protein of the invention to be administered alone, it is desirable to present it as a pharmaceutical formulation, together with one or more acceptable carriers. The carrier(s) must be “acceptable” in the sense of being compatible with the albumin fusion protein and not deleterious to the recipients thereof. Typically, the carriers will be water or saline which will be sterile and pyrogen free. Albumin fusion proteins of the invention are particularly well suited to formulation in aqueous carriers such as sterile pyrogen free water, saline or other isotonic solutions because of their extended shelf-life in solution. For instance, pharmaceutical compositions of the invention may be formulated well in advance in aqueous form, for instance, weeks or months or longer time periods before being dispensed.

Formulations containing the albumin fusion protein may be prepared taking into account the extended shelf-life of the albumin fusion protein in aqueous formulations. As discussed above, the shelf-life of many of these Therapeutic proteins are markedly increased or prolonged after fusion to HA.

In instances where aerosol administration is appropriate, the albumin fusion proteins of the invention can be formulated as aerosols using standard procedures. The term “aerosol” includes any gas-borne suspended phase of an albumin fusion protein of the instant invention which is capable of being inhaled into the bronchioles or nasal passages. Specifically, aerosol includes a gas-borne suspension of droplets of an albumin fusion protein of the instant invention, as may be produced in a metered dose inhaler or nebulizer, or in a mist sprayer. Aerosol also includes a dry powder composition of a compound of the instant invention suspended in air or other carrier gas, which may be delivered by insufflation from an inhaler device, for example.

The formulations may conveniently be presented in unit dosage form and may be prepared by any of the methods well known in the art of pharmacy. Such methods include the step of bringing into association the albumin fusion protein with the carrier that constitutes one or more accessory ingredients. In general the formulations are prepared by uniformly and intimately bringing into association the active ingredient with liquid carriers or finely divided solid carriers or both, and then, if necessary, shaping the product.

Formulations suitable for parenteral administration include aqueous and non-aqueous sterile injection solutions which may contain anti-oxidants, buffers, bacteriostats and solutes which render the formulation appropriate for the intended recipient; and aqueous and non-aqueous sterile suspensions which may include suspending agents and thickening agents. The formulations may be presented in unit-dose or multi-dose containers, for example sealed ampoules, vials or syringes, and may be stored in a freeze-dried (lyophilised) condition requiring only the addition of the sterile liquid carrier, for example water for injections, immediately prior to use. Extemporaneous injection solutions and suspensions may be prepared from sterile powders. Dosage formulations may contain the Therapeutic protein portion at a lower molar concentration or lower dosage compared to the non-fused standard formulation for the Therapeutic protein given the extended serum half-life exhibited by many of the albumin fusion proteins of the invention.

As an example, when an albumin fusion protein of the invention comprises one or more of the Therapeutic protein regions, the dosage form can be calculated on the basis of the potency of the albumin fusion protein relative to the potency of the Therapeutic protein, while taking into account the prolonged serum half-life and shelf-life of the albumin fusion proteins compared to that of the native Therapeutic protein. For example, in an albumin fusion protein consisting of a full length HA fused to a full length Therapeutic protein, an equivalent dose in terms of units would represent a greater weight of agent but the dosage frequency can be reduced.

Formulations or compositions of the invention may be packaged together with, or included in a kit with, instructions or a package insert referring to the extended shelf-life of the albumin fusion protein component. For instance, such instructions or package inserts may address recommended storage conditions, such as time, temperature and light, taking into account the extended or prolonged shelf-life of the albumin fusion proteins of the invention. Such instructions or package inserts may also address the particular advantages of the albumin fusion proteins of the inventions, such as the ease of storage for formulations that may require use in the field, outside of controlled hospital, clinic or office conditions. As described above, formulations of the invention may be in aqueous form and may be stored under less than ideal circumstances without significant loss of therapeutic activity.

The invention also provides methods of treatment and/or prevention of diseases or disorders (such as, for example, any one or more of the diseases or disorders disclosed herein) by administration to a subject of an effective amount of an albumin fusion protein of the invention or a polynucleotide encoding an albumin fusion protein of the invention (“albumin fusion polynucleotide”) in a pharmaceutically acceptable carrier.

Effective dosages of the albumin fusion protein and/or polynucleotide of the invention to be administered may be determined through procedures well known to those in the art which address such parameters as biological half-life, bioavailability, and toxicity, including using data from routine in vitro and in vivo studies, using methods well known to those skilled in the art.

The albumin fusion protein and/or polynucleotide will be formulated and dosed in a fashion consistent with good medical practice, taking into account the clinical condition of the individual patient (especially the side effects of treatment with the albumin fusion protein and/or polynucleotide alone), the site of delivery, the method of administration, the scheduling of administration, and other factors known to practitioners. The “effective amount” for purposes herein is thus determined by such considerations.

For example, determining an effective amount of substance to be delivered can depend upon a number of factors including, for example, the chemical structure and biological activity of the substance, the age and weight of the patient, the precise condition requiring treatment and its severity, and the route of administration. The frequency of treatments depends upon a number of factors, such as the amount of albumin fusion protein or polynucleotide constructs administered per dose, as well as the health and history of the subject. The precise amount, number of doses, and timing of doses will be determined by the attending physician or veterinarian.

Albumin fusion proteins and polynucleotides of the present invention can be administered to any animal, preferably to mammals and birds. Preferred mammals include humans, dogs, cats, mice, rats, rabbits sheep, cattle, horses and pigs, with humans being particularly preferred.

As a general proposition, the albumin fusion protein of the invention will be dosed lower (on the molar basis of the unfused Therapeutic protein) or administered less frequently than the unfused Therapeutic protein. The albumin fusion proteins of the invention are advantageous in that they can simulate continuous infusion of “classic drugs”, i.e., less protein equivalent is needed for identical inhibitory activity.

Albumin fusion proteins and/or polynucleotides can be are administered orally, rectally, parenterally, intracisternally, intravaginally, intraperitoneally, topically (as by powders, ointments, gels, drops or transdermal patch), bucally, or as an oral or nasal spray. “Pharmaceutically acceptable carrier” refers to a non-toxic solid, semisolid or liquid filler, diluent, encapsulating material or formulation auxiliary of any. The term “parenteral” as used herein refers to modes of administration which include intravenous, intramuscular, intraperitoneal, intrasternal, subcutaneous and intra-articular injection and infusion.

Albumin fusion proteins and/or polynucleotides of the invention are also suitably administered by sustained-release systems such as those described in WO 03/066824 and WO 01/79480 (pp. 129-130), which are incorporated herein by reference.

For parenteral administration, in one embodiment, the albumin fusion protein and/or polynucleotide is formulated generally by mixing it at the desired degree of purity, in a unit dosage injectable form (solution, suspension, or emulsion), with a pharmaceutically acceptable carrier, i.e., one that is non-toxic to recipients at the dosages and concentrations employed and is compatible with other ingredients of the formulation. For example, the formulation optionally does not include oxidizing agents and other compounds that are known to be deleterious to the Therapeutic.

The albumin fusion proteins and/or polynucleotides of the invention may be administered alone or in combination with other therapeutic agents. Albumin fusion protein and/or polynucleotide agents that may be administered in combination with the albumin fusion proteins and/or polynucleotides of the invention include, but are not limited to, antiretroviral agents like protease, reverse transcriptase, integrase and assembly inhibitors, chemotherapeutic agents, antibiotics, steroidal and non-steroidal anti-inflammatories, conventional immunotherapeutic agents, and/or therapeutic treatments as described, e.g., in WO 03/066824 and WO 01/79480 (pp. 132-151) which are incorporated by reference herein. Combinations may be administered either concomitantly, e.g., as an admixture, separately but simultaneously or concurrently; or sequentially. This includes presentations in which the combined agents are administered together as a therapeutic mixture, and also procedures in which the combined agents are administered separately but simultaneously, e.g., as through separate intravenous lines into the same individual. Administration “in combination” further includes the separate administration of one of the compounds or agents given first, followed by the second.

Pharmaceutical compositions suitable for use in the present invention include compositions wherein the active ingredients are contained in an effective amount to achieve its intended purpose. In certain embodiments, albumin fusion proteins and/or polynucleotides of the invention are administered in combination with antiretroviral agents, nucleoside/nucleotide reverse transcriptase inhibitors (NRTIs), non-nucleoside reverse transcriptase inhibitors (NNRTIs), and/or protease inhibitors (PIs).

The invention also provides a pharmaceutical pack or kit comprising one or more containers filled with one or more of the ingredients of the pharmaceutical compositions comprising albumin fusion proteins of the invention. Optionally associated with such container(s) can be a notice in the form prescribed by a governmental agency regulating the manufacture, use or sale of pharmaceuticals or biological products, which notice reflects approval by the agency of manufacture, use or sale for human administration.

Having generally described the invention, the same will be more readily understood by reference to the following examples, which are provided by way of illustration and are not intended as limiting.

Without further description, it is believed that one of ordinary skill in the art can, using the preceding description and the following illustrative examples, make and utilize the alterations detected in the present invention and practice the claimed methods. The following working examples therefore, specifically point out certain embodiments of the present invention, and are not to be construed as limiting in any way the remainder of the disclosure.

### EXAMPLE 1

### Preparation of Albumin-Fused IL-11

The recombinant albumin expression vectors pAYE645 and pAYE646 have been described previously in WO 2004/009819. Plasmid pAYE645 contained the HSA/MFα-1 fusion leader sequence, as well as the yeast PRB1 promoter and the yeast ADH1 terminator providing appropriate transcription promoter and transcription terminator sequences, is described in WO 2004/009819. Plasmid pAYE645 was digested to completion with the restriction enzyme AflII and partially digested with the restriction enzyme HindIII and the DNA fragment comprising the 3′ end of the yeast PRB1 promoter and the albumin coding sequence was isolated. Plasmid pDB2241, described in patent application WO 00/44772, was digested with AflII/HindIII and the DNA fragment comprising the 5′ end of the yeast PRPB1 promoter and the yeast ADH1 terminator was isolated. The AflII/HindIII DNA fragment from pAYE645 was then cloned into the AflII/HindIII pDB2241 vector DNA fragment to create the plasmid pDB2302. Plasmid pDB2302 was digested to completion with PacI/XhoI and the 6.19 kb fragment isolated, the recessed ends were blunt ended with T4 DNA polymerase and dNTPs, and religated to generate plasmid pDB2465. Plasmid pDB2465 was linearised with ClaI, the recessed ends were blunt ended with T4 DNA polymerase and dNTPs, and religated to generate plasmid pDB2533. Plasmid pDB2533 was linearised with BlnI, the recessed ends were blunt ended with T4 DNA polymerase and dNTPs, and religated to generate plasmid pDB2534. Plasmid pDB2534 was digested to completion with BmgBI/BglII, the 6.96 kb DNA fragment isolated and ligated to one of two double stranded oligonucleotide linkers, VC053/VC054 and VC057/VC058 to create plasmid pDB2540, or VC055/VC056 and VC057/VC058 to create plasmid pDB2541.

VC053:

(SEQ ID NO: 1)

5′-GATCTTTGGATAAGAGAGACGCTCACAAGTCCGAAGTCGCT-
CACCGGT-3′
VC054:

(SEQ ID NO: 2)

5′-pCCTTGAACCGGTGAGCGACTTCGGACTTGTGAGCGTCTCT-
CTTATCCAAA-3′
VC055:

(SEQ ID NO: 3)

5′-GATCTTTGGATAAGAGAGACGCTCACAAGTCCGAAGTCG-
CTCATCGAT-3′
VC056:

(SEQ ID NO: 4)

5′-pCCTTGAATCGATGAGCGACTTCGGACTTGTGAGCGTCTCTCT-
TATCCAAA-3′
VC057:

(SEQ ID NO: 5)

5′-pTCAAGGACCTAGGTGAGGAAAACTTCAAGGCTTTGGTCT-
TGATCGCTTTCGCTCAATACTTGCAACAATGTCCATTCGAAGATCAC-3′
VC058:

(SEQ ID NO: 6)

5′-GTGATCTTCGAATGGACATTGTTGCAAGTATTGAGCGAAAGCGA-
TCAAGACCAAAGCCTTGAAGTTTTCCTCACCTAGGT-3′

A sample of a human IL11 cDNA was serially diluted from 100 ng.mL⁻¹ to 10 pg.mL⁻¹ (in 10 fold increments). PCR primers CF59 (SEQ ID No: 7) and CF60 (SEQ ID No: 12) were designed to allow the IL11 cDNA to be cloned as an N-terminal albumin fusion into pDB2541 linearised with Bgm and ClaI, whilst at the same time deleting the codon encoding the N-terminal proline from the IL11 coding region. The DNA sequence of each primer were as follows:

```
CF59
                                HSA/MFα-1
              BglII fusion leader IL11
5′-CGATAGATCTTTGGATAAGAGAGGGCCACCACCTGGCCCCC-
CTCGAGTTTCCCC-3′
CF60
                  ClaI albumin IL11
5′-GGCCATCGATGAGCGACTTCGGACTTGTGAGCGTCCAGCCGAGT-
CTTCAGCAGCAGCAGTCCCCTC-3′
```

A master mix was prepared as follows: 2 mM MgCl₂ PCR Buffer, 10 μM PCR dNTP's, 0.2 μM CF59, 0.2 μM CF60, 2U FastStart Taq. DNA polymerase. 1 μL of the IL11 cDNA (10 pg, 100 pg, 1 ng, 10 ng, 100 ng) was added to 49 μL of reaction mix. The total reaction volume was 50 μL. Perkin-Elmer Thermal Cycler 9600 was programmed as follows: Denature at 95° C. for 4 mins [HOLD], then [CYCLE] denature at 95° C. for 30 s, anneal for 30 s at 45° C., extend at 72° C. for 60 s for 20 cycles, followed by a [HOLD] 72° C. for 600 s and then [HOLD] 4° C. The products of the PCR amplification were analysed by gel electrophoresis and a band of expected size (0.59 kb) was observed. The 0.59 kb DNA fragment was isolated from the 1% (w/v) agarose TAE gel using Gene Clean III Kit (BIO101 Inc.).

The PCR DNA fragment was digested to completion with the restriction endonucleases BglII/ClaI and the 0.58 kb fragment was ligated into the 6.15 kb pDB2541 BglII/ClaI vector DNA fragment to create plasmid pDB2567.

Appropriate yeast vector sequences were provide by a “disintegration” plasmid pSAC35 generally disclosed in EP-A-286 424 and described by Sleep, D., et al. (1991) Bio/Technology 9, 183-187. The 3.52 kb NotI N-terminal (des-pro) IL11-albumin expression cassette was isolated from pDB2567, purified and ligated into NotI digested pSAC35 which had been treated with calf intestinal phosphatase, creating plasmid pDB2569 contained the NotI expression cassette in the same orientation to the LEU2 selection marker, and plasmid pDB2570 contained the NotI expression cassette in the opposite orientation to the LEU2 selection marker.

PCR primers CF61 and CF62 were designed to allow the IL11 cDNA to be cloned as a C-terminal albumin fusion into pDB2243 linearised with Bsu36I and partially digested with HindIII, whilst at the same time adding two “TAA” translation stop codons at the 3′ end of the IL11 open reading frame. Plasmid pDB2243, previously described in patent application WO 00/44772, which contained the yeast PRB1 promoter and the yeast ADH1 terminator provided appropriate transcription promoter and transcription terminator sequences. The DNA sequence of the CF61 (SEQ ID No: 13) and CF62 (SEQ ID No: 14) primers was as follows:

```
CF61
              Bsu36I albumin IL11
5′-CCGGCCTTAGGCTTACCTGGGCCACCACCTGGCCCCCCTCG-
AGTTTCCCC-3′
CF62
              HindIII IL11
5′-GGCCAAGCTTATTACAGCCGAGTCTTCAGCAGCAGCAGTCCCCTC-
3′
                  STOP STOP
```

A master mix was prepared as follows: 2 mM MgCl₂ PCR Buffer, 10 μM PCR dNTP's, 0.2 μM CF61, 0.2 μM CF62, 2U FastStart Taq. DNA polymerase. 1 μL of the IL11 cDNA (10 pg, 100 pg, 1 ng, 10 ng, 100 ng) was added to 49 μL of reaction mix. The total reaction volume was 50 μL. Perkin-Elmer Thermal Cycler 9600 was programmed as follows: Denature at 95° C. for 4 mins [HOLD], then [CYCLE] denature at 95° C. for 30 s, anneal for 30 s at 45° C., extend at 72° C. for 60 s for 20 cycles, followed by a [HOLD] 72° C. for 600 s and then [HOLD] 4° C. The products of the PCR amplification were analysed by gel electrophoresis and a band of expected size (0.55 kb) was observed. The 0.59 kb DNA fragment was isolated from the 1% (w/v) agarose TAE gel using Gene Clean III Kit (BIO101 Inc.).

The PCR DNA fragment was digested to completion with the restriction endonucleases Bsu36I/HindIII and the 0.55 kb fragment was ligated into the 6.19 kb pDB2243 Bsu36I, partially digested with HindIII vector DNA fragment to create plasmid pDB2568.

Appropriate yeast vector sequences were provide by a “disintegration” plasmid pSAC35 generally disclosed in EP-A-286 424 and described by Sleep, D., et al. (1991) Bio/Technology 9, 183-187. The 3.53 kb NotI C-terminal albumin-IL11 expression cassette was isolated from pDB2568, purified and ligated into NotI digested pSAC35 which had been treated with calf intestinal phosphatase, creating plasmid pDB2571 contained the NotI expression cassette in the same orientation to the LEU2 selection marker, and plasmid pDB2572 contained the NotI expression cassette in the opposite orientation to the LEU2 selection marker. Yeast strains disclosed in WO 95/23857, WO 95/33833 and WO 94/04687 were transformed to leucine prototrophy as described in Sleep D., et al. (2001) Yeast 18, 403-421. The transformants were patched out onto Buffered Minimal Medium (BMM, described by Kerry-Williams, S. M. et al. (1998) Yeast 14, 161-169) and incubated at 30° C. until grown sufficiently for further analysis.

DNA sequence of the N-terminal IL11-albumin fusion open reading frame forms SEQ ID No: 15.

Amino acid sequence of the N-terminal IL11-albumin fusion protein is presented as SEQ ID No: 16.

Amino acid sequence of the mature N-terminal IL11-albumin fusion protein forms SEQ ID No: 17.

GPPPGPPRVSPDPRAELDSTVLLTRSLLADTRQLAAQLRDKFPADGDHNL
DSLPTLAMSAGALGALQLPGVLTRLRADLLSYLRHVQWLRRAGGSSLKTL
EPELGTLQARLDRLLRRLQLLMSRLALPQPPPDPPAPPLAPPSSAWGGIR
AAHAILGGLHLTLDWAVRGLLLLKTRLDAHKSEVAHRFKDLGEENFKALV
LIAFAQYLQQCPFEDHVKLVNEVTEFAKTCVADESAENCDKSLHTLFGDK
LCTVATLRETYGEMADCCAKQEPERNECFLQHKDDNPNLPRLVRPEVDVM
CTAFHDNEETFLKKYLYEIARRHPYFYAPELLFFAKRYKAAFTECCQAAD
KAACLLPKLDELRDEGKASSAKQRLKCASLQKFGERAFKAWAVARLSQRF
PKAEFAEVSKLVTDLTKVHTECCHGDLLECADDRADLAKYICENQDSISS
KLKECCEKPLLEKSHCIAEVENDEMPADLPSLAADFVESKDVCKNYAEAK
DVFLGMFLYEYARRHPDYSVVLLLRLAKTYETTLEKCCAAADPHECYAKV
FDEFKPLVEEPQNLIKQNCELFEQLGEYKFQNALLVRYTKKVPQVSTPTL
VEVSRNLGKVGSKCCKHPEAKRMPCAEDYLSVVLNQLCVLHEKTPVSDRV
TKCCTESLVNRRPCFSALEVDETYVPKEFNAETFTFHADICTLSEKERQI
KKQTALVELVKHKPKATKEQLKAVMDDFAAFVEKCCKADDKETCFAEEGK
KLVAASQAALGL

DNA sequence of the C-terminal albumin-IL11 fusion open reading frame is SEQ ID No: 18.

And the corresponding amino acid sequence of the C-terminal albumin-IL11 fusion protein is SEQ ID No: 19.

The amino acid sequence of the mature C-terminal albumin-IL11 fusion protein is SEQ ID No: 20.

### EXAMPLE 2

### Purification

### C-Terminal IL11 Purification

The C-Terminal IL11 fusion contained high levels of clipped (i.e. not full length) material. It was purified using the standard rHA SP-FF conditions as described in WO 00/44772 but in a negative mode whereby the fusion was in the flowthrough. The flowthrough was adjusted to pH 8 and 2.5 mS.cm⁻¹ and loaded on a standard rHA DE-FF equilibrated in 15 mM potassium tetraborate. This was operated in a negative mode. The conductivity of the DE-FF flowthrough was increased to 15 mS.cm⁻¹ and the material purified using standard rHA DBA chromatography with an extra elution of 50 mM octanoate in the equilibration buffer. The material was then concentrated and diafiltered against 300 mM glycine, 10 mM phosphate pH7.

N-Terminal IL11 Purification (type A)

The N-Terminal IL11 contained some clipped material. It was purified using the standard rHA SP-FF conditions as described in WO 00/44772. The majority was in the flowthrough but sufficient bound for it to be necessary for it to be eluted using the standard elution buffer containing 200 mM NaCl. The eluate was then adjusted to pH 8 and 2.5 mS.cm⁻¹ and purified using standard rHA DE-FF equilibrated in 15 mM potassium tetraborate. The DE-FF was eluted using the standard rHA elution buffer. The purified material was then concentrated and diafiltered against 300 mM glycine, 10 mM phosphate pH7.

### N-Terminal IL11 Purification (Type B)

The N-terminal IL-11 fusion protein contained some clipped material. This material was separated using a S-Sepharose-FF column according to the standard rHA SP-FF conditions. Free rHA remained on the column. Then the fusion protein in the flowthrough was adsorbed to a monoclonal antibody Sepharose specific for albumin. The column was washed with high salt and eluted at pH 2.5. The eluate was adjusted to neutral pH, concentrated and diafiltered against 300 mM glycine, 10 mM Na-phosphate pH 7.0.

### Protein Characterisation After Purification

TABLE 1
IL-11 albumin fusion characterisation

| | | | N-Terminal |
|---|---|---|---|
| | C-Terminal | N-Terminal | Fusion (Type |
| | Fusion | Fusion (Type A) | B) |
| % Purity by | 77 | 90 | 89 |
| SDS-PAGE and | | | |
| colloidal blue | | | |
| staining | | | |
| ESMS | Theoretical | Theoretical | Theoretical |
| indication | mass 85565. | mass 85468. | mass 85468. |
| of post- | Measured | Measured mass | Measured mass |
| translational | mass 68000- | 85471. Good | 85471. Good |
| modifications | 70000. | evidence for | evidence for |
| | | correct primary | correct primary |
| | | structure. | structure. |
| N-Terminal | N/A | Correct NT | |
| Sequence | | sequence for | |
| | | IL 11. | |
| Endotoxin | 73 | 23 | 130 |
| (EU · mL⁻¹) | | | |
| Fusion | 1.5 | 2.7 | 1 |
| Concentration | | | |
| (mg · mL⁻¹) | | | |

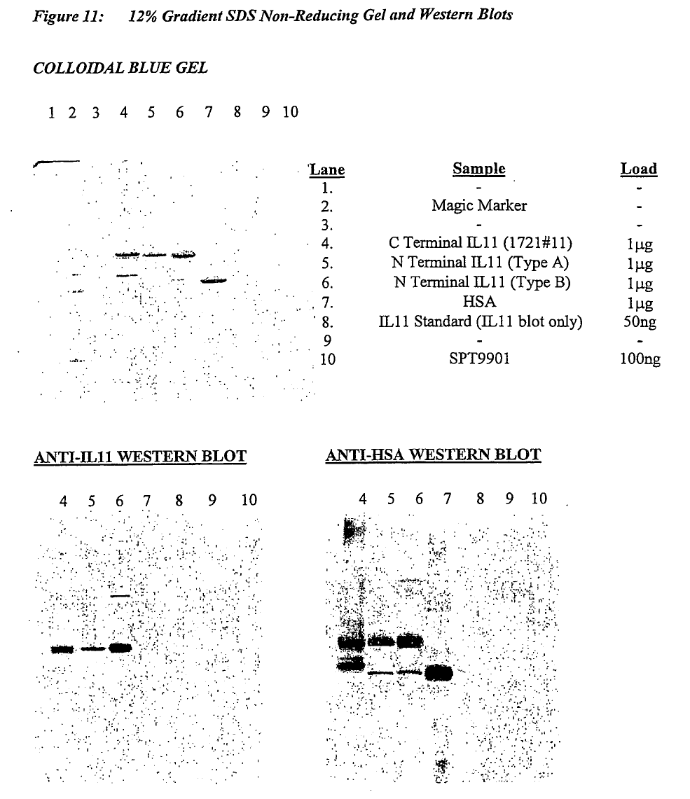

Images of the 12% Gradient SDS Non-Reducing Gel and Western Blots are shown in FIG. 11.

### EXAMPLE 3

### Pharmacokinetics of Albumin-Fused IL-11 Versus Recombinant Human IL-11 After Single Intravenous or Subcutaneous Administration to Rabbits

Three male and three female rabbits per group received Neumega® IL-11 (100 μg/kg) or C-terminal albumin-fused IL-11 (440 μg/kg) by a single i.v. or s.c. injection on day 0 (Table 2). Blood samples were drawn for the determination of the respective antigen levels at baseline and at 5 min, 10 min, 20 min, 30 min, 45 min, 1 h, 2 h, 4 h, 8 h, 24 h (1 d), 48 h (2 d), 72 h (3 d), 5 d, 7 d, 9 d, 11 d, and 14 d after i.v. administration of the respective test substance and at baseline, 30 min, 1 h, 2 h, 4 h, 8 h, 24 h (1 d), 48 h (2 d), 72 h (3 d), 5 d, 7 d, 9 d, 11 d and 14 d following s.c. injection. The doses of Neumega® IL-11 and C-terminal albumin-fused IL-11 were calculated on an equimolar basis.

### Measurement of IL-11 Plasma Levels

Plasma levels of human IL-11 were measured by Quantikine® Human IL-11 Immunoassay (R&D Systems, Catalog No. D1100). This assay employs the quantitative sandwich enzyme immunoassay technique. A murine monoclonal antibody specific to IL-11 has been coated onto a microplate. Standards and samples are pipetted into the wells and any IL-11 present is bound by the immobilized antibody. After washing away any unbound substances, an enzyme-linked polyclonal antibody specific for IL-11 is added to the wells. Following a wash to remove any unbound antibody-enzyme reagent, a substrate solution is added to the wells and colour develops in proportion to the amount of IL-11 bound in the initial step. The colour development is stopped and the intensity of the colour is measured.

Plasma Half Life of Fused Vs. Unfused Protein in Rabbits

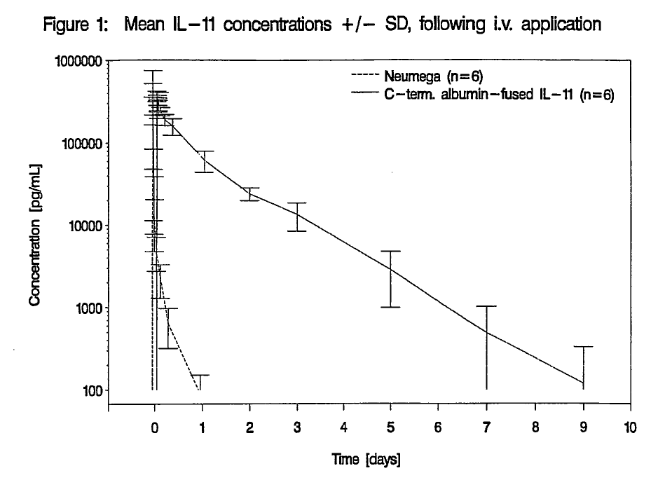

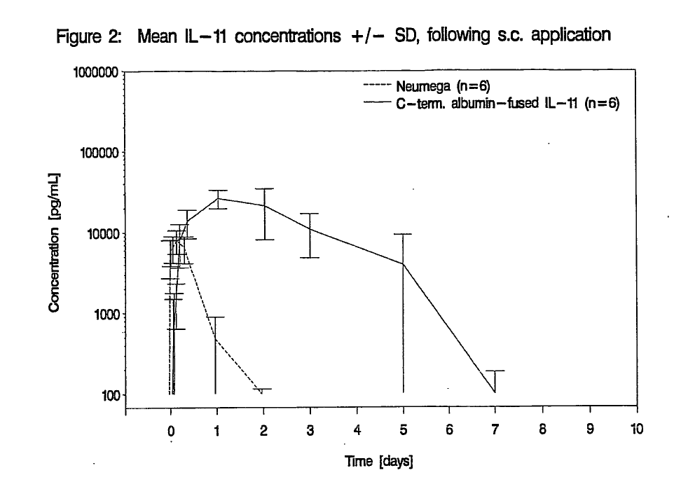

The means and standard deviations of the IL-11 concentrations at every time point are shown in FIG. 1 for the i.v.-treated groups and in FIG. 2 for the s.c. treated groups.

In the animals treated intravenously, levels above 100 pg/mL can be found for 1 day after injection in the Neumega® IL-11 group and for 9 days after injection in the albumin-fused IL-11 group.

In the animals treated subcutaneously, the levels in the Neumega® IL-11 group reach their peak around 6 hours after injection and stay above 100 pg/mL for little less than 2 days (FIG. 2). The levels in the albumin-fused IL-11 group reach their peak 24 hours after injection and stay above 100 pg/mL for just under 7 days.

The pharmacokinetic results are presented in Table 2 for the i.v.-treated groups and in Table 3 for the s.c.-treated groups.

TABLE 2
Pharmacokinetic results following i.v. administration

| | | Neumega ® | C-terminal albumin- |
|---|---|---|---|
| | | IL-11 | fused IL-11 |
| | N | 6 | 6 |
| Initial half-life | Mean | 0.12 | 3.61 |
| (hr) | Std Dev | 0.03 | 2.53 |
| | Median | 0.11 | 3.32 |
| | Range | 0.09-0.15 | 0.30-6.52 |
| Terminal half- | Mean | 3.31 | 22.79 |
| life (hr) | Std Dev | 2.30 | 8.07 |
| | Median | 2.12 | 22.08 |
| | Range | 1.59-7.01 | 14.73-35.93 |
| AUC₀₋₁₄ | Mean | 162,686 | 5,421,764 |
| (hr · pg/mL) | Std Dev | 60,920 | 513,997 |
| | Median | 156,014 | 5,552,217 |
| | Range | 99,915-232,051 | 4,719,147-5,946,916 |
| | Geometric | 152,862 | 5,397,976 |
| | mean | | |
| | Scatter | 1.47 | 1.10 |
| | factor* | | |
| Cₘₐₓ (pg/mL) | Mean | 547,425 | 395,308 |
| | Std Dev | 194,193 | 61,216 |
| | Median | 540,825 | 379,975 |
| | Range | 274,200-760,450 | 332,300-489,100 |
| Total clearance | Mean | 721 | 21.0 |
| (mL/hr/kg) | Std Dev | 335 | 3.1 |
| | Median | 662 | 19.5 |
| | Range | 382-1,123 | 18.5-19.5 |
| Total volume of | Mean | 771 | 537 |
| distribution | Std Dev | 636 | 170 |
| (mL/kg) | Median | 628 | 500 |
| | Range | 204-1,781 | 377-847 |
| Mean residence | Mean | 0.94 | 25.1 |
| time (hr) | Std Dev | 0.49 | 4.3 |
| | Median | 0.81 | 25.2 |
| | Range | 0.44-1.63 | 20.4-31.9 |

*Scatter factor = exp[standard deviation (log-transformed values)]

TABLE 3
Pharmacokinetic results following s.c. administration

| | | Neumega ® | C-terminal albumin- |
|---|---|---|---|
| | | IL-11 | fused IL-11 |
| | N | 6 | 6 |
| Absorption | Mean | 1.14 | 16.5 |
| half-life (hr) | Std Dev | 0.76 | 8.5 |
| | Median | 0.81 | 15.0 |
| | Range | 0.35-2.12 | 7.7-32.5 |
| Terminal half- | Mean | 4.67 | 18.0 |
| life (hr) | Std Dev | 1.43 | 3.9 |
| | Median | 4.40 | 19.2 |
| | Range | 3.07-6.67 | 11.7-22.3 |
| AUC₀₋₁₄ | Mean | 122,093 | 1,794,152 |
| (hr · pg/mL) | Std Dev | 28,159 | 753,505 |
| | Median | 118,860 | 1,665,227 |
| | Range | 95,376-172,688 | 1,113,203-3,000,848 |
| | Geometric | 119,635 | 1,670,725 |
| | mean | | |
| | Scatter | 1.24 | 1.51 |
| | factor* | | |
| Cₘₐₓ (pg/mL) | Mean | 8,098 | 30,590 |
| | Std Dev | 2,468 | 9,091 |
| | Median | 8,061 | 29,420 |
| | Range | 5,342-11,117 | 21,261-44,730 |
| tₘₐₓ (hr) | Mean | 5.67 | 28.0 |
| | Std Dev | 2.66 | 9.80 |
| | Median | 6.00 | 24.0 |
| | Range | 2.00-8.00 | 24.0-48.0 |
| Relative total | Mean | 1,270 | 70.9 |
| clearance | Std Dev | 407 | 27.5 |
| (mL/hr/kg) | Median | 1,286 | 69.9 |
| | Range | 639-1,842 | 38.3-102.3 |
| Relative total | Mean | 8,880 | 1753 |
| volume of | Std Dev | 4,969 | 532 |
| distribution | | | |
| (mL/kg) | Median | 6,910 | 1,599 |
| | Range | 4,512-15,836 | 1,041-2,495 |

*Scatter factor = exp[standard deviation (log-transformed values)}

### Subcutaneous Versus Intravenous Administration in Rabbits

C-terminal albumin-fused IL-11 showed an average elimination half-life that was 8 times longer than that of Neumega® IL-11 after i.v. application (Table 4). The area under the curve was 35 times larger. After s.c. injection, the average elimination half-life of C-terminal albumin-fused IL-11 was 4 times longer than that of Neumega® IL-11. The area under the curve was 14 times larger.

TABLE 4
Comparison of bioavailability between substances

| | | Elimination | | |
|---|---|---|---|---|
| Route | Parameter | half-life | AUC₀₋₁₄ | Cₘₐₓ |
| i.v. | Estimated ratio (C-terminal | 7.85 | 35.3 | 0.76 |
| | albumin-fused IL-11/ | | | |
| | Neumega ®) | | | |
| | 90% confidence limits | 5.24-11.76 | 25.9-48.2 | 0.56-1.03 |
| s.c. | Estimated ratio (C-terminal | 3.93 | 14.0 | 3.80 |
| | albumin-fused IL-11/ | | | |
| | Neumega ®) | | | |
| | 90% confidence limits | 2.62-5.89 | 10.2-19.1 | 2.79-5.17 |

The bioavailability of Neumega® s.c. versus i.v. was 78% (Table 5) while for C-terminal albumin fusion it was calculated as 31%.

TABLE 5
Comparison of bioavailability between routes of application

| | | Elimination | | |
|---|---|---|---|---|
| Substance | Parameter | half-life | AUC₀₋₁₄ | Cₘₐₓ |
| Neumega ® | Estimated ratio | 1.63 | 0.78 | 0.015 |
| IL-11 | (s.c./i.v.) | | | |
| | 90% confidence | 1.09-2.44 | 0.57-1.07 | 0.011-0.021 |
| | limits | | | |
| C-term. | Estimated ratio | 0.81 | 0.31 | 0.075 |
| albumin- | (s.c./i.v.) | | | |
| fused IL-11 | 90% confidence | 0.54-1.22 | 0.23-0.42 | 0.055-0.103 |
| | limits | | | |

### EXAMPLE 4

### Pharmacokinetics of Albumin-Fused IL-11 Versus Recombinant Human IL-11 After Intravenous or Subcutaneous Administration to Rats

Three rats per group received Neumega® IL-11 (100 μg/kg) or N-terminal albumin-fused IL-11 (Type A) (440 μg/kg) by a single i.v. or s.c. injection on day 0 (Table 6). Blood samples for the determination of the respective antigen levels were drawn at the following timepoints: pre-injection, 2 min, 5 min, 10 min, 15 min, 30 min, 45 min, 1 h, 2 h, 8 h, 24 h, 48 h, 72 h (3 d), 5 d, 7 d. The doses of Neumega® IL-11 and N-terminal albumin-fused IL-11 were calculated on an equimolar basis.

### Study Design

### Measurement of IL-11 Plasma Levels

Plasma levels for groups 1 and 3 (Neumega® IL-11) were measured with an anti-human IL-11 ELISA by Quantikine® Human IL-11 Immunoassay (R&D Systems, Catalog No. D1100), as described in Example 3 above. Plasma levels for groups 2 and 4 (IL-11-AFP) were determined with an anti-human albumin ELISA. The standard for this assay was IL-11-AFP.

Plasma Half Life of Fused Vs. Unfused Protein in Rats

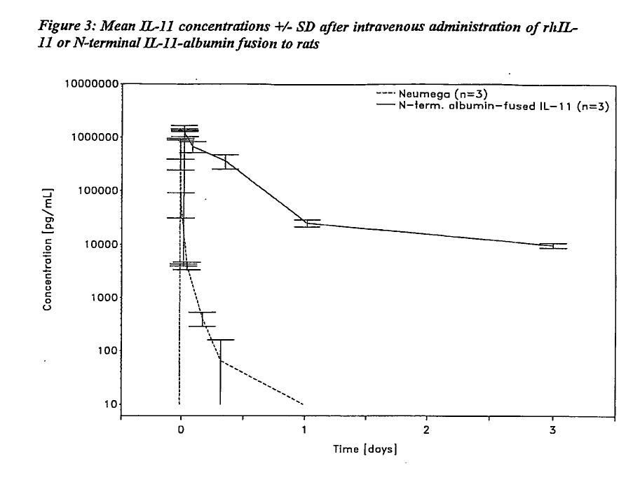

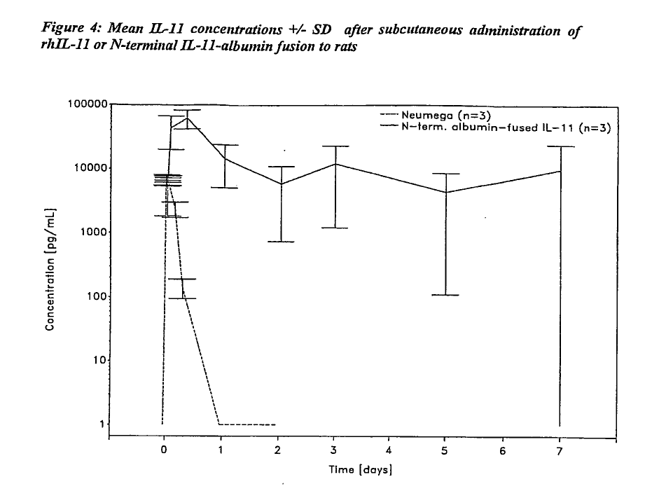

In the animals treated intravenously (FIG. 3), levels above 0.1 ng/mL were found for 6 to 12 hours after injection in the Neumega® group and for 3 days after injection in the albumin-fused IL-11 group. In the animals treated subcutaneously (FIG. 4), the levels in the Neumega® IL-11 group reached their peak within 1 hour after injection and stayed above 0.1 ng/mL for about 9 hours. The levels in the albumin-fused IL-11 group reached their peak between 2 and 8 hours after injection and stayed above 0.1 ng/mL for at least 44 hours.

The pharmacokinetic results are presented in Table 6 for the i.v. treated groups and in Table 7 for the s.c. treated groups.

TABLE 6
Pharmacokinetic results following i.v. administration

| | | Neumega ® | N-terminal albumin- |
|---|---|---|---|
| | | Il-11 | fused IL-11 |
| | N | 3 | 3 |
| Initial half- | Mean | 0.13 | 3.15 |
| life (hr) | Std Dev | 0.09 | 2.45 |
| | Median | 0.09 | 3.09 |
| | Range | 0.07-0.24 | 0.73-5.62 |
| Terminal | Mean | 1.39 | 28.08 |
| half-life | Std Dev | 0.92 | 23.10 |
| (hr) | Median | 0.92 | 24.57 |
| | Range | 0.80-2.46 | 6.93-52.73 |
| AUC | Mean | 26,375 | 8,357,724 |
| (hr · pg/mL) | Std Dev | 4,595 | 1,214,981 |
| | Median | 25,108 | 8,164,280 |
| | Range | 22,548-31,471 | 7,251,070-9,657,822 |
| | Geometric | 26,118 | 8,299,777 |
| | mean | | |
| | Scatter | 1.19 | 1.15 |
| | factor* | | |
| Cₘₐₓ (pg/mL)** | Mean | 33,510 | 1,065,644 |
| | Std Dev | 4,310 | 133,261 |
| | Median | 31,565 | 1,134,432 |
| | Range | 30,515-38,450 | 912,045-1,150,455 |
| Total | Mean | 4,324 | 14.1 |
| clearance | Std Dev | 570 | 2.2 |
| (mL/hr/kg) | Median | 4,253 | 15.1 |
| | Range | 3,793-4,926 | 11.6-15.7 |
| Total volume | Mean | 3,281 | 157 |
| of | Std Dev | 538 | 27 |
| distribution | Median | 2,971 | 150 |
| (mL/kg) | Range | 2,970-3,902 | 134-187 |
| Mean | Mean | 0.78 | 11.2 |
| residence time | Std Dev | 0.22 | 2.1 |
| (hr) | Median | 0.70 | 11.9 |
| | Range | 0.60-1.03 | 8.9-12.9 |

*Scatter factor = exp[standard deviation (log-transformed values)]
**measured at 10 minutes, the earliest post-injection measurement in study PSR April 2002

TABLE 7
Pharmacokinetic results following s.c. administration

| | | Neumega ® | N-terminal albumin- |
|---|---|---|---|
| | | Il-11 | fused IL-11 |
| | N | 3 | 3 |
| Absorption | Mean | 0.74 | 2.63 |
| half-life (hr) | Std Dev | 0.19 | 1.97 |
| | Median | 0.85 | 3.24 |
| | Range | 0.52-0.85 | 0.42-4.22 |
| Terminal | Mean | 0.87 | 6.70 |
| half-life (hr) | Std Dev | 0.04 | 4.84 |
| | Median | 0.86 | 4.66 |
| | Range | 0.85-0.92 | 3.21-12.23 |
| AUC | Mean | 24,687 | 1,032,955 |
| (hr · pg/mL) | Std Dev | 2,547 | 335,512 |
| | Median | 25,706 | 1,043,068 |
| | Range | 21,788-26,566 | 692,500-1,363,295 |
| | Geometric | 24,596 | 994,888 |
| | mean | | |
| | Scatter | 1.11 | 1.41 |
| | factor* | | |
| Cₘₐₓ (pg/mL) | Mean | 7,550 | 63,636 |
| | Std Dev | 135 | 14,205 |
| | Median | 7,612 | 62,159 |
| | Range | 7,395-7,642 | 50,227-78,523 |
| tₘₐₓ (hr) | Mean | 1.0 | 6.0 |
| | Std Dev | 0.0 | 3.5 |
| | Median | 1.0 | 8.0 |
| | Range | 1.0-1.0 | 2.0-8.0 |
| Relative total | Mean | 4,168 | 112.0 |
| clearance | Std Dev | 480 | 47.5 |
| (mL/hr/kg) | Median | 3,962 | 87.9 |
| | Range | 3,826-4,718 | 81.3-166.6 |
| Relative total | Mean | 5,275 | 956 |
| volume of | Std Dev | 842 | 527 |
| distribution | Median | 4,858 | 771 |
| (mL/kg) | Range | 4,723-6,244 | 547-1,550 |

*Scatter factor = exp[standard deviation (log-transformed values)]

The maximum IL-11 concentrations measured in Study PSR 01/03 are presented in Table 8.

TABLE 8
Maximum values obtained within one hour post-injection

| | | | Neumega ® | N-terminal albumin-fused |
|---|---|---|---|---|
| | | | IL-11 | IL-11 |
| | | N | 3 | 3 |
| i.v. | Cₘₐₓ | Mean | 910,200 | 1,537,879 |
| | (pg/mL) | Std Dev | 30,524 | 109,122 |
| | | Median | 911,600 | 1,581,818 |
| | | Range | 879,000-940,000 | 1,413,636-1,618,182 |
| s.c. | Cₘₐₓ | Mean | 7,086 | 0 |
| | (pg/mL) | Std Dev | 618 | 0 |
| | | Median | 7,133 | 0 |
| | | Range | 6,445-7,679 | 0-0 |
| | tₘₐₓ | Mean | 0.42 | — |
| | (hours) | Std Dev | 0.29 | — |
| | | Median | 0.25 | — |
| | | Range | 0.25-0.75 | 0.25-1.00 |

### Subcutaneous Versus Intravenous Administration in Rats

Table 9 shows the results of the analyses of variance regarding the relative bioavailability. The differences between the two products were significant with respect to elimination half-life, AUC and Cₘₐₓ for both routes of application.

TABLE 9
Comparison of bioavailability between substances

| | | Elimination | | |
|---|---|---|---|---|
| Route | Parameter | half-life | AUC | Cₘₐₓ |
| i.v. | Estimated ratio | 17.03 | 317.8 | 1.69 |
| | (N-terminal | | | |
| | albumin-fused IL-11/ | | | |
| | Neumega ®) | | | |
| | 90% confidence limits | 5.98-48.49 | 230.8-437.5 | 1.41-2.02 |
| s.c. | Estimated ratio | 6.50 | 40.5 | 8.29 |
| | (N-terminal | | | |
| | albumin-fused IL-11/ | | | |
| | Neumega ®) | | | |
| | 90% confidence limits | 2.28-18.50 | 29.4-55.7 | 6.92-9.93 |

Table 10 shows the results of the analyses of variance regarding the absolute bioavailability. For Neumega® IL-11, the differences between the two routes of application were not statistically significant with respect to elimination half-life and AUC. The difference in Cₘₐₓ was highly significant. For albumin-fused IL-11, the differences between the two routes of application were not significant with respect to elimination half-life. The differences regarding AUC and Cₘₐₓ were highly significant.

TABLE 10
Comparison of bioavailability between routes of application

| | | Elimination | | |
|---|---|---|---|---|
| Substance | Parameter | half-life | AUC | Cₘₐₓ^{a} |
| Neumega ® | Estimated | 0.72 | 0.942 | 0.008 |
| IL-11 | ratio | | | |
| | (s.c./i.v.) | | | |
| | 90% confi- | 0.25-2.04 | 0.684-1.296 | 0.007-0.010 |
| | dence limits | | | |
| N-term. | Estimated | 0.27 | 0.120 | 0.041 |
| albumin- | ratio | | | |
| | (s.c./i.v.) | | | |
| fused | 90% confi- | 0.10-0.78 | 0.087-0.165 | 0.034-0.049 |
| IL-11 | dence limits | | | |

^{a}Cₘₐₓ values of i.v. route taken from Study PSR January 2003, measured 2 minutes post-injection.

In rats N-terminal albumin-fused IL-11 showed an average elimination half-life that was 17 times longer than that of Neumega® IL-11 after i.v. application. The area under the curve was 318 times larger. After s.c. injection, the average elimination half-life of N-terminal albumin-fused IL-11 was 6 times longer than that of Neumega® IL-11. The area under the curve was 40 times larger.

### EXAMPLE 5

### Stimulation of Thrombocytopoiesis in Naive Rats by IL11-Fusion Protein

Naive CD-rats (10 per group) were treated with Neumega® IL-11, C-terminal- or N-terminal-fused IL-11 (type B), or placebo according to the following schedule.

TABLE 11
Treatment schedule

| Group | Substance | Dose | Appl. Sch. | Vol. | N |
|---|---|---|---|---|---|
| 1 | Formulation | — | t = d 0-d 9 s.c. | 0.5 ml | 10 |
| | solution | | | | |
| 2 | Neumega ® | 100 μg/kg | t = d 0-d 9 s.c. | 0.5 ml | 10 |
| | IL-11 | | | | |
| 3 | IL-11-AFP | 660 μg/kg | t = d 0, d 3, d 6, | 0.5 ml | 10 |
| | C-term | | d 9 s.c. | | |
| 4 | IL-11-AFP | 660 μg/kg | t = d 0, d 3, d 6, | 0.5 ml | 10 |
| | N-term | | d 9 s.c. | | |
| 5 | IL-11-AFP | 660 μg/kg | t = d 0-d 9 s.c. | 0.5 ml | 10 |
| | C-term | | | | |
| 6 | IL-11-AFP | 660 μg/kg | t = d 0-d 9 s.c. | 0.5 ml | 10 |
| | N-term | | | | |

Blood samples were drawn at baseline, day 5, day 7, day 9, day 13 and day 16 and hematologic parameters (PLT, WBC, RBC, HCT, HGB) and body weight were measured. The dose of the fusion protein was calculated on an equimolar basis compared to Neumega® IL-11 and corrected by a factor of 1.5 due to SDS-PAGE results.

### Results

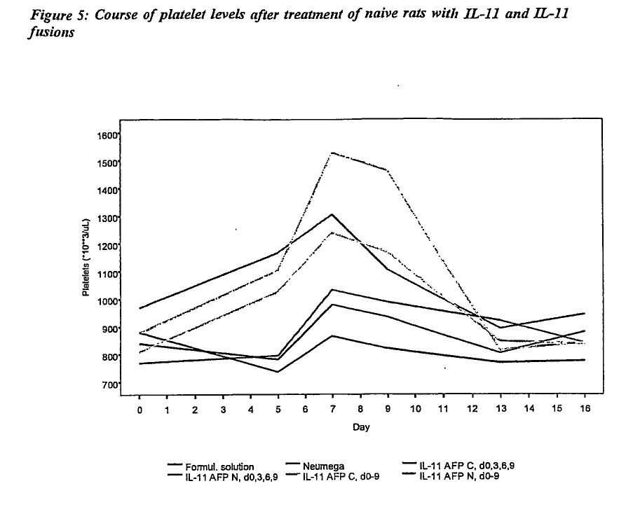

Maximum platelet counts were achieved in all groups on day 7 (FIG. 5). The highest mean levels of 1528×103/μL were achieved with the N-terminal fusion administered daily (group 6) or every three days (group 4) with 1304×103/μL. The C-terminal fusion also showed a dose-interval related effect with maximum mean levels of 1238×103/μL when administered daily compared to 977×103/μL if given every three days. Neumega® IL-11 reached peak levels of 1032×103/μL while the control animals remained at 864×103/μL.

Red blood parameters (RBC, HGB, HCT) remained constant for all groups. White blood cell counts showed a slight initial increase followed by a non-consistent fluctuation. This can probably be attributed to the microtrauma caused by frequent injections and blood sampling. All animals showed a normal, slight increase in body weight over the course of the observation period.

In summary, both the N- and the C-terminal fusion administered daily as well as the N-terminal fusion administered in 3-day-intervals were clearly superior to control and Neumega® IL-11 on days 5, 7 and 9. However, the N-terminal fusion seems to achieve higher levels than the C-terminal fusion.

The decrease of platelet counts after day 7 despite continuation of treatment until day 9 might occur due to the development of neutralizing antibodies to the heterologous human protein.

### EXAMPLE 6

### Treatment of Chemotherapy-Induced Thrombocytopenia in Rats by IL-11-Fusion-Protein

Ten female CD-rats per group received Carboplatin at 35 mg/kg i.v. on day 0. Starting on day 5 they were treated with Neumega® IL-11, C-terminal- or N-terminal-fused IL-11 (type B), or placebo according to the following schedule.

TABLE 12
Treatment schedule

| Group | Substance | Dose | Appl. Sch. | Vol. | N |
|---|---|---|---|---|---|
| 1 | Formulation | — | t = d 5-d 14 s.c. | 0.5 ml | 10 |
| | solution | | | | |
| 2 | Neumega ® | 50 μg/kg | t = d 5-d 14 s.c. | 0.5 ml | 10 |
| 3 | IL-11-AFP | 330 μg/kg | t = d 5, d 8, d 11, d | 0.5 ml | 10 |
| | C-term | | 14 s.c. | | |
| 4 | IL-11-AFP | 330 μg/kg | t = d 5, d 8, d 11, d | 0.5 ml | 10 |
| | N-term | | 14 s.c. | | |
| 5 | IL-11-AFP | 330 μg/kg | t = d 5-d 14 s.c. | 0.5 ml | 10 |
| | C-term | | | | |
| 6 | IL-11-AFP | 330 μg/kg | t = d 5-d 14 s.c. | 0.5 ml | 10 |
| | N-term | | | | |

Blood samples were drawn at baseline, day 5, day 7, day 9, day 13 and day 16 and hematologic parameters (PLT, WBC, RBC, HCT, HGB) and body weight were measured. The dose of the fusion protein was calculated on an equimolar basis compared to Neumega® IL-11 and corrected by a factor of 1.5 due to SDS-PAGE results.

### Results

In all groups platelet nadirs were observed between day 5 and day 9 (Table 13). Thereafter platelet levels recovered to above baseline on day 16. Mean platelet levels are shown in the following table.

TABLE 13
Mean platelet levels (×10³/μL)

| | Group 1 | Group 2 | Group 3 | Group 4 | Group 5 | Group 6 |
|---|---|---|---|---|---|---|
| day 0 | 782 | 783 | 788 | 703 | 813 | 810 |
| day 5 | 477 | 281 | 368 | 458 | 344 | 365 |
| day 7 | 388 | 302 | 303 | 330 | 348 | 374 |
| day 9 | 365 | 323 | 365 | 375 | 328 | 432 |
| day 13 | 603 | 702 | 681 | 832 | 689 | 1064 |
| day 16 | 1003 | 1034 | 969 | 974 | 901 | 1287 |

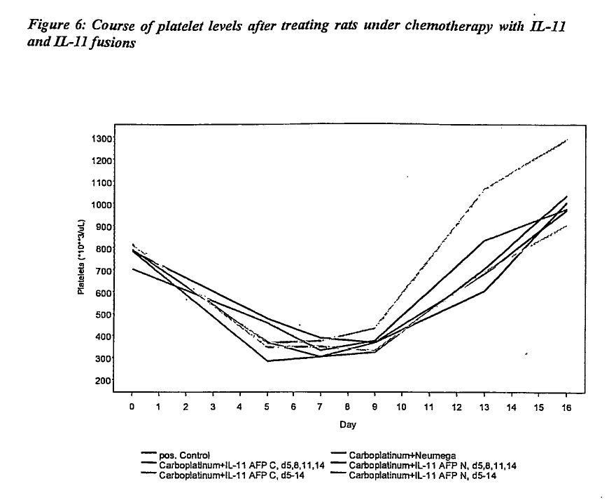

For Neumega® IL-11, C- and N-terminal fusion injected daily, the platelet-nadir was observed on day 5, the first day of treatment (FIG. 6). Thereafter in the group receiving the N-terminal fusion daily the platelet levels increased steadily and significantly, while the C-terminal fusion administered daily induced an increase comparable to Neumega® IL-11.

An analysis of covariance adjusted for the day 5 platelet value shows a significantly higher platelet count for N-terminal fusion injected daily as compared to control on days 9, 13 and 16, as well as significant superiority over Neumega® IL-11 on day 13. The same analysis demonstrates an overall treatment effect with p≦0.004 on days 7, 13 and 16.

Considering the duration of platelet levels below 500×10³/μL again the N-terminal fusion shows the best effects at a mean of 5.87 days when injected daily or 6.27 every 3 days as compared to 8.08 days for Neumega® IL-11 and 6.93 days for controls. The C-terminal fusion shows less efficacy in that respect with a duration of 7.16 days with daily administration and 6.69 every 3 days.

Red blood parameters (RBC, HGB, HCT) showed a slight decrease during the study period in all groups, which might reflect the blood loss caused by frequent blood sampling. White blood cell counts showed a slight increase which can probably be attributed to the microtrauma caused by frequent injections and blood sampling. However this effect was especially prominent in the animals receiving N-terminal fusion of IL-11 daily, so that in this group a treatment related effect on WBC levels can not be excluded. All animals showed a normal, slight increase in body weight over the course of the observation period.

In summary the most prominent effect can be observed with the N-terminal albumin fusion of IL-11, while the C-terminal fusion behaves comparable to Neumega® IL-11. Even though the absolute levels at nadir are not affected significantly, the N-terminal fusion protein is able to reduce the duration of levels below 500×10³/μL and leads to a significantly steeper recovery as compared to Neumega® IL-11.

### EXAMPLE 7

### IL-11-Fusion Protein in a Mouse Model of Inflammatory Bowel Disease (IBD)

In this model colitis is induced in mice through oral administration of 3% dextran sulfate disodium salt (DSS) in tap water. Ten mice per group were allocated to the following treatment groups.

TABLE 14
Treatment schedule

| Group | Treatment | Dose | Appl scheme | Volume | n |
|---|---|---|---|---|---|
| 1 | Formulation solu- | n.a. | t = d 0-9 s.c.; | 0.2 mL | 10 |
| | tion, no DSS | | 1×/day | | |
| 2 | Formulation | n.a. | t = d 0-9 s.c.; | 0.2 mL | 10 |
| | solution | | 1×/day | | |
| 3 | Sulfasalazine | 100 mg/kg | t = d 0-10; p.o. | ad lib. | 10 |
| 4 | Neumega ® | 250 μg/kg | t = d 0-9 s.c.; | 0.2 mL | 10 |
| | IL-11 | | 1×/day | | |
| 5 | IL-11-AFP | 1650 μg/kg | t = d 0, 3, 6, 9 | 0.2 mL | 10 |
| | C-term | | s.c.; 1×/day | | |
| 6 | IL-11-AFP | 1650 μg/kg | t = d 0, 3, 6, 9 | 0.2 mL | 10 |
| | N-term (type B) | | s.c.; 1×/day | | |

Body weight, rectal bleeding/diarrhoea and occult blood in faeces were assessed before induction and on days 3, 6 and 9. The experiment was terminated on day 10, the animals were necropsied, colon length was measured and samples of large intestine were retained for histological evaluation.

### Results

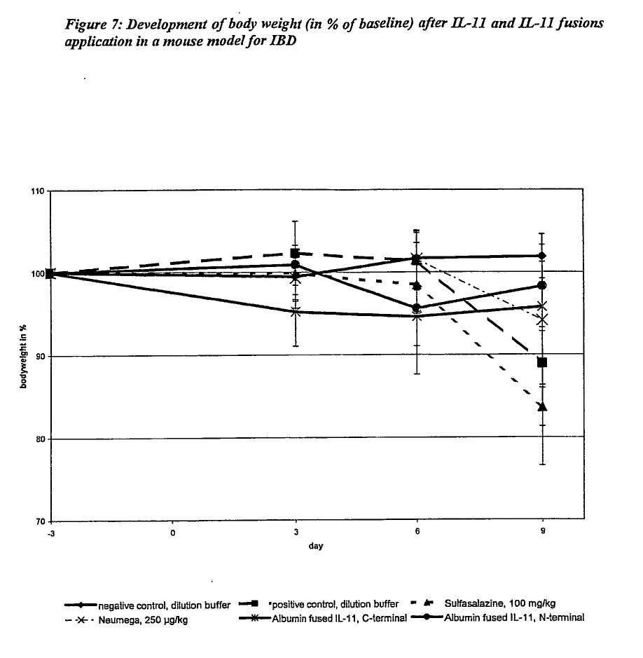

Animals receiving placebo, Sulfasalazine or Neumega® IL-11 lost weight after the onset of colitis, while the animals treated with N- or C-terminal fusion even gained weight (FIG. 7). This was a surprising effect that could not have been predicted in advance.

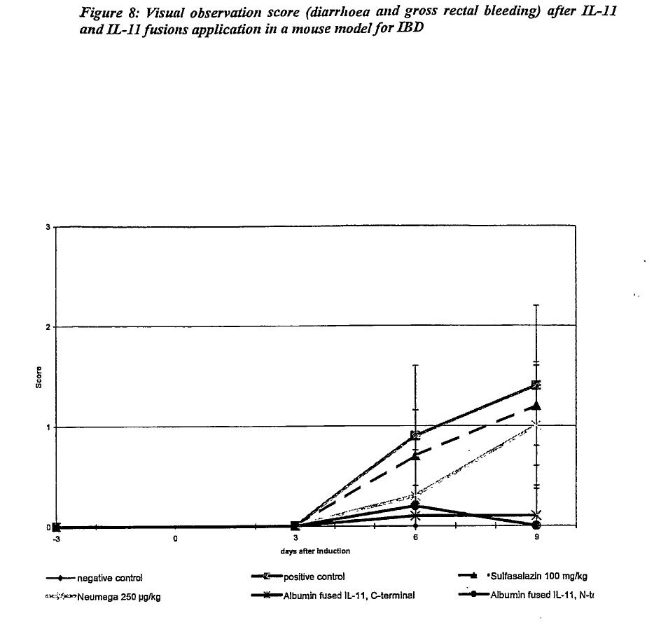

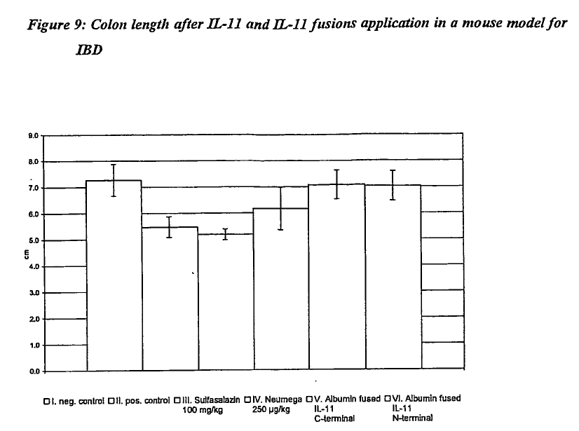

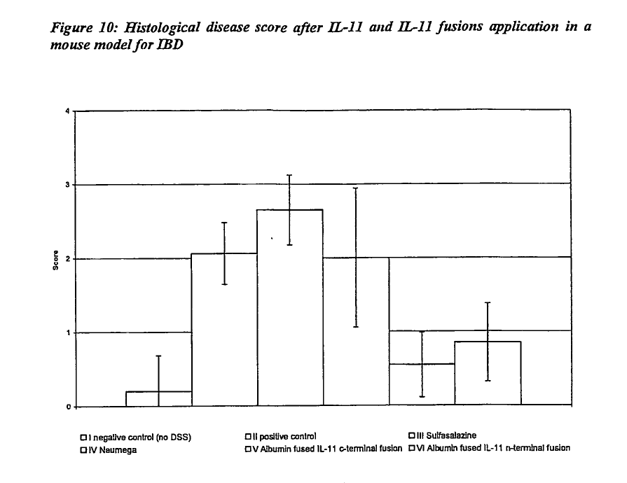

The visual observation score evaluating diarrhoea and rectal bleeding also shows that there was a surprising, and significant, beneficial effect of the fusion proteins compared to that of placebo, Sulfasalazine or Neumega® IL-11 (FIG. 8). The same holds true for the length of the colon (FIG. 9) and the histological score (FIG. 10).

In summary these data show that both albumin fusions given in 3 day intervals are able to significantly ameliorate the symptoms of DSS-induced colitis in mice, even superior to Neumega® IL-11 or the current standard treatment Sulfasalazine.

### EXAMPLE 8

### Shelf-Life

Shelf-life of the albumin-fused IL-11 products was determined according to the following method. A total of 25 mg of the albumin-fused IL-11 products was formulated to a final volume of 5 ml in a glass vial in sterile water-for-injection, with glycine, heptahydrate di-basic sodium phosphate, and mono-basic basic sodium phosphate as excipients Vials of the products were incubated at 25° C. for 1 month. Samples were assayed at the end of one month as described in Example 1 for post-translational modifications, by ESMS, N-terminal sequence, non-reducing SDS polyacrylamide gels and Western blots. Shelf-life is considered to be prolonged if the compound exhibits fewer changes, in any one of these tests, after this storage than the unfused IL-11 compound.

### EXAMPLE 9

### Human Administration

At least in the case of the treatment of cancers, the albumin-fused IL-11 products may be administered by single s.c. injection once every four days, with a dose regime of 25-1000 μg per kg body weight, preferably 25-50 μg per kg.

### REFERENCES

The present invention is not to be limited in scope by the specific embodiments described which are intended as single illustrations of individual aspects of the invention, and functionally equivalent methods and components are within the scope of the invention. Indeed, various modifications of the invention, in addition to those shown and described herein will become apparent to those skilled in the art from the foregoing description and accompanying drawings. Such modifications are intended to fall within the scope of the appended claims. Every reference cited hereinabove is incorporated by reference in its entirety.

1. An albumin fusion protein comprising IL-11, or a fragment or variant thereof, and albumin, or a fragment or variant thereof.

2. The albumin fusion protein of claim 1, wherein the IL-11 is human IL-11.

3. An albumin fusion protein according to claim 1, comprising an albumin fused to IL-11.

4. The albumin fusion protein of claim 1, wherein the IL-11 is human IL-11.

5. The albumin fusion protein of claim 1 wherein the albumin has the ability to prolong the in vivo half-life of IL-11, or a fragment or variant thereof, compared to the in vivo half-life of IL-11, or a fragment or variant thereof, in an unfused state.

6. The protein of claim 5, whereby the half-life of said albumin-fused IL-11 is extended at least 5-fold over the half-life of the IL-11 lacking the linked albumin.

7. The protein of claim 6, whereby the half-life of said albumin-fused IL-11 is extended at least 10-fold over the half-life of the IL-11 lacking the linked albumin.

8. The protein of claim 7 whereby the half-life of said albumin-fused IL-11 is extended at least 50-fold over the half-life of the IL-11 lacking the linked albumin.

9. The albumin fusion protein of claim 1 wherein IL-11, or a fragment or variant thereof, is fused to the N-terminus of albumin, or the N-terminus of the fragment or variant of albumin.

10. The albumin fusion protein of claim 1 wherein IL-11, or a fragment or variant thereof, is fused to the C-terminus of albumin, or the C-terminus of the fragment or variant of albumin.

11. The albumin fusion protein of claim 1 wherein IL-11, or a fragment or variant thereof, is fused to an internal region of albumin, or an internal region of a fragment or variant of albumin.

12. The albumin fusion protein of claim 1 wherein IL-11, or a fragment or variant thereof, is separated from the albumin or the fragment or variant of albumin by a linker.

13. The albumin fusion protein of claim 1 wherein the in vitro biological activity of the IL-11, or fragment or variant thereof, fused to albumin, or fragment or variant thereof, is greater than the in vitro biological activity IL-11, or fragment or variant thereof, in an unfused state.

14. The albumin fusion protein of claim 1 wherein the in vivo biological activity of IL-11, or fragment or variant thereof, fused to albumin, or fragment or variant thereof, is greater than the in vivo biological activity of IL-11, or fragment or variant thereof, in an unfused state.

15. A nucleic acid molecule comprising a polynucleotide sequence encoding the albumin fusion protein of claim 1.

16. A vector comprising the nucleic acid molecule of claim 15.

17. A host cell containing the nucleic acid molecule of claim 15.

18. A method for manufacturing an albumin fusion protein of claim 1, the method comprising (a) providing a nucleic acid comprising a nucleotide sequence encoding the albumin fusion protein expressible in a cell or organism; (b) expressing the nucleic acid in the cell or organism to form an albumin fusion protein; and (c) purifying the albumin fusion protein.

19. The method of claim 18 wherein the albumin fusion protein is expressed in a yeast.

20. The method of claim 19 wherein the yeast is glycosylation deficient.

21. The method of claim 19 wherein the yeast is glycosylation competent.

22. The method of claim 18 wherein the albumin fusion protein is expressed in a mammalian cell in cell culture.

23. A composition comprising the albumin fusion protein of claim 1 and a carrier.

24. A pharmaceutical composition comprising an effective amount of the albumin fusion protein of claim 1 and a pharmaceutically acceptable carrier or excipient.

25. A method for minimizing a side effect associated with the treatment of a mammal with IL-11 comprising administering an albumin-fused IL-11 to said mammal.

26. A method according to claim 25 wherein the mammal is suffering from a bowel disorder and the side effect is weight loss, rectal bleeding or diarrhoea.

27. A method of increasing weight in a mammal suffering from a bowel disease causing weight loss, the method comprising administering an albumin-fused IL-11 to said mammal.

28. A method of treating a disease or disorder in a patient, comprising the step of administering an effective amount of the albumin fusion protein of claim 1.

29. A method of treating a patient, comprising the step of administering an effective amount of the albumin fusion protein of claim 1.

30. A method of extending the in vivo half-life of IL-11, or a fragment or variant thereof, comprising the step of fusing IL-11, or fragment or variant thereof, to albumin or a fragment or variant of albumin sufficient to extend the in vivo half-life of IL-11, or fragment or variant thereof, compared to the in vivo half-life of IL-11, or fragment or variant thereof, in an unfused state.

31. A method for extending the half-life of IL-11 in a mammal, the method comprising linking said IL-11 to an albumin to form an albumin-fused IL-11 and administering said albumin-fused IL-11 to said mammal, whereby the half-life of said albumin-fused IL-11 is extended at least 2-fold over the half-life of IL-11 lacking the linked albumin.

32. A method for preventing or treating thrombocytopenia in a mammal, the method comprising administering an albumin-fused IL-11 to said mammal.

33. A method for minimizing a side effect associated with the treatment of a mammal with IL-11, the method comprising administering an albumin-fused IL-11 to said mammal.


SEQUENCE LISTING (20 sequences)

SEQ ID NO: 1 - DNA, 48 bp, Artificial Sequence
  Oligonucleotide Linker VC053
gatctttggataagagagacgctcacaagtccgaagtcgctcaccggt

SEQ ID NO: 2 - DNA, 50 bp, Artificial Sequence
  Oligonucleotide Linker VC054
ccttgaaccggtgagcgacttcggacttgtgagcgtctctcttatccaaa

SEQ ID NO: 3 - DNA, 48 bp, Artificial Sequence
  Oligonucleotide Linker VC055
gatctttggataagagagacgctcacaagtccgaagtcgctcatcgat

SEQ ID NO: 4 - DNA, 50 bp, Artificial Sequence
  Oligonucleotide Linker VC056
ccttgaatcgatgagcgacttcggacttgtgagcgtctctcttatccaaa

SEQ ID NO: 5 - DNA, 86 bp, Artificial Sequence
  Oligonucleotide Linker VC057
tcaaggacctaggtgaggaaaacttcaaggctttggtcttgatcgctttcgctcaatacttgcaacaatgtccattcgaagatcac

SEQ ID NO: 6 - DNA, 80 bp, Artificial Sequence
  Oligonucleotide Linker VC058
gtgatcttcgaatggacattgttgcaagtattgagcgaaagcgatcaagaccaaagccttgaagttttcctcacctaggt

SEQ ID NO: 7 - DNA, 54 bp, Artificial Sequence
  Oligonucleotide Primer CF59
cgatagatctttggataagagagggccaccacctggcccccctcgagtttcccc

SEQ ID NO: 8 - protein, 5 aa, Artificial Sequence
  Polypeptide linker SEQ ID 8
GGGGS

SEQ ID NO: 9 - protein, 4 aa, Artificial Sequence
  Polypeptide linker SEQ ID 9
GGGS

SEQ ID NO: 10 - protein, 17 aa, Artificial Sequence
  Stanniocalcin signal peptide sequence
MLQNSAVLLLLVISASA

SEQ ID NO: 11 - protein, 22 aa, Artificial Sequence
  Consensus signal sequence
MPTWAWWLFLVLLLALWAPARG

SEQ ID NO: 12 - DNA, 66 bp, Artificial Sequence
  Oligonucleotide Primer CF60
ggccatcgatgagcgacttcggacttgtgagcgtccagccgagtcttcagcagcagcagtcccctc

SEQ ID NO: 13 - DNA, 50 bp, Artificial Sequence
  Oligonucleotide Primer CF61
ccggccttaggcttacctgggccaccacctggcccccctcgagtttcccc

SEQ ID NO: 14 - DNA, 45 bp, Artificial Sequence
  Oligonucleotide Primer CF62
ggccaagcttattacagccgagtcttcagcagcagcagtcccctc

SEQ ID NO: 15 - DNA, 2358 bp, Artificial Sequence
  N-terminal IL11-albumin fusion
atgaagtgggttttcatcgtctccattttgttcttgttctcctctgcttactctagatctttggataagagagggccaccacctggcccccctcgagtttccccagaccctcgggccgagctggacagcaccgtgctcctgacccgctctctcctggcggacacgcggcagctggctgcacagctgagggacaaattcccagctgacggggaccacaacctggattccctgcccaccctggccatgagtgcgggggcactgggagctctacagctcccaggtgtgctgacaaggctgcgagcggacctactgtcctacctgcggcacgtgcagtggctgcgccgggcaggtggctcttccctgaagaccctggagcccgagctgggcaccctgcaggcccgactggaccggctgctgcgccggctgcagctcctgatgtcccgcctggccctgccccagccacccccggacccgccggcgcccccgctggcgcccccctcctcagcctgggggggcatcagggccgcccacgccatcctgggggggctgcacctgacacttgactgggccgtgaggggactgctgctgctgaagactcggctggacgctcacaagtccgaagtcgctcatcgattcaaggacctaggtgaggaaaacttcaaggctttggtcttgatcgctttcgctcaatacttgcaacaatgtccattcgaagatcacgtcaagttggtcaacgaagttaccgaattcgctaagacttgtgttgctgacgaatctgctgaaaactgtgacaagtccttgcacaccttgttcggtgataagttgtgtactgttgctaccttgagagaaacctacggtgaaatggctgactgttgtgctaagcaagaaccagaaagaaacgaatgtttcttgcaacacaaggacgacaacccaaacttgccaagattggttagaccagaagttgacgtcatgtgtactgctttccacgacaacgaagaaaccttcttgaagaagtacttgtacgaaattgctagaagacacccatacttctacgctccagaattgttgttcttcgctaagagatacaaggctgctttcaccgaatgttgtcaagctgctgataaggctgcttgtttgttgccaaagttggatgaattgagagacgaaggtaaggcttcttccgctaagcaaagattgaagtgtgcttccttgcaaaagttcggtgaaagagctttcaaggcttgggctgtcgctagattgtctcaaagattcccaaaggctgaattcgctgaagtttctaagttggttactgacttgactaaggttcacactgaatgttgtcacggtgacttgttggaatgtgctgatgacagagctgacttggctaagtacatctgtgaaaaccaagactctatctcttccaagttgaaggaatgttgtgaaaagccattgttggaaaagtctcactgtattgctgaagttgaaaacgatgaaatgccagctgacttgccatctttggctgctgacttcgttgaatctaaggacgtttgtaagaactacgctgaagctaaggacgtcttcttgggtatgttcttgtacgaatacgctagaagacacccagactactccgttgtcttgttgttgagattggctaagacctacgaaactaccttggaaaagtgttgtgctgctgctgacccacacgaatgttacgctaaggttttcgatgaattcaagccattggtcgaagaaccacaaaacttgatcaagcaaaactgtgaattgttcgaacaattgggtgaatacaagttccaaaacgctttgttggttagatacactaagaaggtcccacaagtctccaccccaactttggttgaagtctctagaaacttgggtaaggtcggttctaagtgttgtaagcacccagaagctaagagaatgccatgtgctgaagattacttgtccgtcgttttgaaccaattgtgtgttttgcacgaaaagaccccagtctctgatagagtcaccaagtgttgtactgaatctttggttaacagaagaccatgtttctctgctttggaagtcgacgaaacttacgttccaaaggaattcaacgctgaaactttcaccttccacgctgatatctgtaccttgtccgaaaaggaaagacaaattaagaagcaaactgctttggttgaattggtcaagcacaagccaaaggctactaaggaacaattgaaggctgtcatggatgatttcgctgctttcgttgaaaagtgttgtaaggctgatgataaggaaacttgtttcgctgaagaaggtaagaagttggtcgctgcttcccaagctgctttgggtttg

SEQ ID NO: 16 - protein, 786 aa, Artificial Sequence
  N-terminal IL11-albumin fusion
MKWVFIVSILFLFSSAYSRSLDKRGPPPGPPRVSPDPRAELDSTVLLTRSLLADTRQLAAQLRDKFPADGDHNLDSLPTLAMSAGALGALQLPGVLTRLRADLLSYLRHVQWLRRAGGSSLKTLEPELGTLQARLDRLLRRLQLLMSRLALPQPPPDPPAPPLAPPSSAWGGIRAAHAILGGLHLTLDWAVRGLLLLKTRLDAHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGL

SEQ ID NO: 17 - protein, 762 aa, Artificial Sequence
  Mature N-terminal IL11-albumin fusion
GPPPGPPRVSPDPRAELDSTVLLTRSLLADTRQLAAQLRDKFPADGDHNLDSLPTLAMSAGALGALQLPGVLTRLRADLLSYLRHVQWLRRAGGSSLKTLEPELGTLQARLDRLLRRLQLLMSRLALPQPPPDPPAPPLAPPSSAWGGIRAAHAILGGLHLTLDWAVRGLLLLKTRLDAHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGL

SEQ ID NO: 18 - DNA, 2361 bp, Artificial Sequence
  C-terminal albumin-IL11 fusion
atgaagtgggtaagctttatttcccttctttttctctttagctcggcttattccaggagcttggataaaagagatgcacacaagagtgaggttgctcatcggtttaaagatttgggagaagaaaatttcaaagccttggtgttgattgcctttgctcagtatcttcagcagtgtccatttgaagatcatgtaaaattagtgaatgaagtaactgaatttgcaaaaacatgtgttgctgatgagtcagctgaaaattgtgacaaatcacttcataccctttttggagacaaattatgcacagttgcaactcttcgtgaaacctatggtgaaatggctgactgctgtgcaaaacaagaacctgagagaaatgaatgcttcttgcaacacaaagatgacaacccaaacctcccccgattggtgagaccagaggttgatgtgatgtgcactgcttttcatgacaatgaagagacatttttgaaaaaatacttatatgaaattgccagaagacatccttacttttatgccccggaactccttttctttgctaaaaggtataaagctgcttttacagaatgttgccaagctgctgataaagctgcctgcctgttgccaaagctcgatgaacttcgggatgaagggaaggcttcgtctgccaaacagagactcaagtgtgccagtctccaaaaatttggagaaagagctttcaaagcatgggcagtagctcgcctgagccagagatttcccaaagctgagtttgcagaagtttccaagttagtgacagatcttaccaaagtccacacggaatgctgccatggagatctgcttgaatgtgctgatgacagggcggaccttgccaagtatatctgtgaaaatcaagattcgatctccagtaaactgaaggaatgctgtgaaaaacctctgttggaaaaatcccactgcattgccgaagtggaaaatgatgagatgcctgctgacttgccttcattagctgctgattttgttgaaagtaaggatgtttgcaaaaactatgctgaggcaaaggatgtcttcctgggcatgtttttgtatgaatatgcaagaaggcatcctgattactctgtcgtgctgctgctgagacttgccaagacatatgaaaccactctagagaagtgctgtgccgctgcagatcctcatgaatgctatgccaaagtgttcgatgaatttaaacctcttgtggaagagcctcagaatttaatcaaacaaaattgtgagctttttgagcagcttggagagtacaaattccagaatgcgctattagttcgttacaccaagaaagtaccccaagtgtcaactccaactcttgtagaggtctcaagaaacctaggaaaagtgggcagcaaatgttgtaaacatcctgaagcaaaaagaatgccctgtgcagaagactatctatccgtggtcctgaaccagttatgtgtgttgcatgagaaaacgccagtaagtgacagagtcaccaaatgctgcacagaatccttggtgaacaggcgaccatgcttttcagctctggaagtcgatgaaacatacgttcccaaagagtttaatgctgaaacattcaccttccatgcagatatatgcacactttctgagaaggagagacaaatcaagaaacaaactgcacttgttgagctcgtgaaacacaagcccaaggcaacaaaagagcaactgaaagctgttatggatgatttcgcagcttttgtagagaagtgctgcaaggctgacgataaggagacctgctttgccgaggagggtaaaaaacttgttgctgcaagtcaagctgccttaggcttacctgggccaccacctggcccccctcgagtttccccagaccctcgggccgagctggacagcaccgtgctcctgacccgctctctcctggcggacacgcggcagctggctgcacagctgagggacaaattcccagctgacggggaccacaacctggattccctgcccaccctggccatgagtgcgggggcactgggagctctacagctcccaggtgtgctgacaaggctgcgagcggacctactgtcctacctgcggcacgtgcagtggctgcgccgggcaggtggctcttccctgaagaccctggagcccgagctgggcaccctgcaggcccgactggaccggctgctgcgccggctgcagctcctgatgtcccgcctggccctgccccagccacccccggacccgccggcgcccccgctggcgcccccctcctcagcctgggggggcatcagggccgcccacgccatcctgggggggctgcacctgacacttgactgggccgtgaggggactgctgctgctgaagactcggctg

SEQ ID NO: 19 - protein, 787 aa, Artificial Sequence [sequence omitted]

SEQ ID NO: 20 - protein, 763 aa, Artificial Sequence
  Mature C-terminal IL11-albumin fusion
DAHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGLPGPPPGPPRVSPDPRAELDSTVLLTRSLLADTRQLAAQLRDKFPADGDHNLDSLPTLAMSAGALGALQLPGVLTRLRADLLSYLRHVQWLRRAGGSSLKTLEPELGTLQARLDRLLRRLQLLMSRLALPQPPPDPPAPPLAPPSSAWGGIRAAHAILGGLHLTLDWAVRGLLLLKTRL